(19) United States
(12) Patent Application Publication
(10) Pub. No.: US 2020/0391169 A1
(43) Pub. Date: Dec. 17, 2020

(54) Droplet Microfluidic Synthesis of Electrically Distinct Polymer Particles for Detection, Quantification, and Barcoding

(71) Applicant: The Board of Trustees of the University of Illinois, Urbana, IL (US)

(72) Inventors: Hee-Sun HAN, Champaign, IL (US); Rashid BASHIR, Champaign, IL (US); Thomas COWELL, Houston, TX (US); Enrique VALERA, Champaign, IL (US); Anurup GANGULI, Champaign, IL (US)

(21) Appl. No.: 16/883,244

(22) Filed: May 26, 2020

Related U.S. Application Data
(60) Provisional application No. 62/861,454, filed on Jun. 14, 2019.

(51) Int. Cl. B01J 19/00; G01N 33/543; G01N 33/546; C12Q 1/6813; G01N 15/10

(52) U.S. Cl. CPC B01J 19/0046; G01N 33/54306; G01N 33/546; B01J 2219/00736; C12Q 1/6813; G01N 15/1031; B01J 2219/00599; G01N 33/54393

(57) ABSTRACT

Provided herein are multiplexible particle systems and related methods of making and using the multiplexible particle systems. A plurality of monodisperse polymer particle populations are provided, wherein each population has a unique electrical parameter for multiplexed detection by flow through a spatially confined electric field, and the distribution of the electrical parameter within each population is sufficiently narrow for reliable multiplex detection. The density difference between populations may be relatively uniform, such as within 30%, including within 30% of a suspending solution density for when the particles are flowed through a confined electric field and detected in a multiplex manner by a change in the electric parameter measured by a counting device. Relatively uniform density of particles is important for ensuring minimal settling while the plurality of particle populations flow together under a single flow regime. The multiplexible particle systems are used in applications including multiplex detection or quantification, electrically barcoding, sorting, and counting.

### CROSS-REFERENCE TO RELATED APPLICATIONS

This application claims the benefit of priority to U.S. Provisional Patent Application No. 62/861,454, filed Jun. 14, 2019, which is hereby incorporated by reference in its entirety to the extent not inconsistent herewith.

### BACKGROUND OF INVENTION

Provided herein are systems, methods and related applications related to multiplex detection, relying, at least in part, on electrical parameter measurement of a plurality of polymer particle populations, each population having a unique property, such as a property that affects the measured electrical parameter.

Devices relying on coulter counting and immunocapture to count either a single cell or a single particle are available. See, e.g., U.S. Pat. No. 9,976,973; U.S. Pat. Pub. No. 2018/0364186. Using immunocapture and a second Coulter Counter® allows a single protein target, either on a bead surface or a cell surface, to be quantified using the difference in counts up and downstream from the immunocapture. The ability of this technology to quantitate a single protein from human plasma samples in approximately 10 minutes has been demonstrated. Valera et al. “A microfluidic biochip platform for electrical quantification of proteins.” Lab Chip. 2018 18, 1461, 2018. The problem with those systems, however, is they are not capable of detecting a plurality of different targets or, more generally, a plurality of unique events in a single run. The systems and methods provided herein address this lack of multiplexibilty problem by using electrically distinguishable particle (bead) populations, in a manner where the particle populations retain some uniformity with respect to size, density and corresponding flow properties.

Another technology for electrical quantification of protein utilizes coulter counting of bead aggregates to quantitate a single protein. That technology relies on bead aggregation in the presence of a protein target. The aggregated beads are much larger than the single particles, and so produce a higher impedance signal on a Coulter Counter. That technology is limited compared to the systems described and claimed in this patent application as that technology relies on the full range of impedance intensities to detect and quantify a single protein. The system and methods described herein, in contrast, can use a narrow range of impedances (the distribution of each population) to read out the results of a single biological assay, making it suitable for multiplexing.

There are many existing methods of multiplexed protein detection that use optical components to read and distinguish multiple protein signals. Optical readout requires complex instrumentation which results in higher cost relative to an electrical measurement device. The most prevalent of those technologies rely on an ELISA which uses a primary antibody to capture target proteins from a sample on a solid support. Secondary antibodies which have enzyme labels amplify the protein binding events enzymatically converting many substrate molecules into a detectable chemical signal (fluorescence, chemiluminescence, colorimetry). Multiplexing is typically achieved by using unique enzyme-substrate pairs for each protein target such that the resulting chemical signals produce a non-overlapping optical response.

A similar technology (Luminex xMAP) using dye-labeled beads achieves multiplexing. Each bead has surface antibodies that are determined by the color and intensity of dyes in the bead. Those primary antibodies capture protein targets on the bead. Those binding events are transduced into a detectable signal using secondary antibodies with biotin labels. Fluorescently labeled streptavidin then binds to the biotin on the secondary antibody. Optical detection of each bead reads the streptavidin signal as a measure of the protein concentration, while the bead dyes distinguish the protein identity of each assay.

As a label-free technique, the impedance-based technology provided herein is different from the above-discussed multiplex detection assays in its fundamental simplicity. Instead of using optical and spectrophotometric components to distinguish the wavelength and intensity of light which encode the identity and quantity of analytes, the polymer beads (optionally incorporated with magnetic beads) can fully measure a sample using only the impedance measurements. The intensity of the signal encodes the assay identity while the depletion of the exit counts relative to the entrance provides the quantitative result of the assay. The electrical detection schemes provided herein reduces the cost and complexity of multiplexed protein quantification, and are compatible with relatively simple and robust point-of-care devices.

The problem in the art of lack of reliable and simple platforms that are multiplexible is addressed herein by using a specially configured plurality of polymer cell populations, each having a unique electrical parameter, to achieve, robust, reliable and cost-effective assays, including without a need for optical labels or detection.

### SUMMARY OF THE INVENTION

Disclosed herein are electrically distinguishable beads or particles useful in a range of applications, including for multiplexed detection, quantification, or electrical barcoding and sorting. The introduction of multiplexing capabilities by the electrically distinct particle populations described herein addresses one of the major limiting problems with conventional technology, namely the ability to reliably and robustly perform multiplex detection or quantification in a cost-effective and reliable manner. The multiplex platforms provided herein are compatible with a range of targets and target types, including biomolecules. For example, proteins, cells, polynucleotides (DNA or RNA) are compatible with the instant invention. As but one example, multiplex protein quantification is important for accurate diagnosis and prognosis, as research has found that single protein measurements are less predictive than a correlated measurement of multiple biomarkers. The invention provided herein addresses this need in the art for multiplexing.

Importantly, the particle populations provided herein, while producing unique impedances during detection, can be highly similar in size and density, thereby ensuring flow properties across populations is relatively compatible for a single flow run in a given instrument. This ensures that the particles can be captured uniformly in response to various protein binding assays, thereby enabling multiplexed biomarker detection and quantification.

Polymer particles, such as hydrogel beads and magnetic hydrogel particles, are a unique set of materials having the required properties, such as density, size, composition and/or structure, to produce distinct electrical impedances and achieve various functions. There have been techniques to produce many identical micron-size hydrogel beads using microfluidics, but provided herein is a platform that produces multiple populations of particles/beads that have distinct electrical impedance signatures, which enables multiplex coulter detection. The distinct impedances are achieved without relying on particles that are substantially distinct in terms of size or density. This advancement in microparticle design is important for producing uniform capture dynamics which is necessary for sensitive multiplexed quantification.

The multiple populations of electrically distinguishable polymer particles can be rapidly counted and identified using an electrical measurement of their unique impedances. Importantly, these polymer particles may comprise hydrogel, which has many advantageous material properties. First it has a density near that of water which allows the particles to flow through the device without unwanted settling inside the capture chamber, which would reduce the accuracy and sensitivity of the multiplex assay. Second, hydrogels are cheap to produce. Third, the hydrogel particles are biocompatible and comprise FDA approved materials. Fourth, hydrogels are thermostable across a wide range of temperatures allowing for cheap and simple storage at ambient conditions. Fifth, hydrogels may be formed of degradable materials and so may be broken down readily for easy disposal. Beyond this the hydrogel beads are highly tunable enabling their properties to be tailored to the specific application.

For example, the physical and chemical properties can be varied to achieve different bead properties that are well suited to the situation. We have explored various polymer materials including, polyacrylamide, poly(N-isopropylacrylamide), alginate, agarose, and others to produce hydrogels with different physical and chemical properties. Notably, some of these materials result in hydrogels that are well suited to growing cells, are temperature responsive, undergo pH dependent degradation, or have increased stability and mechanical strength.

We are able to impart chemical functionalization on the particle surface enabling the particles to act as a platform for many distinct biological assays using conjugation chemistries to affix selective biomolecules to the particles. Also, we can incorporate magnetic micro- and nano-particles in the hydrogel material. Magnetic beads can be rapidly separated from solution making washing steps fast, easy and reliable. It is also possible to distinguish magnetic particles from non-magnetic particles with overlapping impedance signatures by counting with and without a magnet applied to the bead solution. As a result of these properties of the electrically distinct hydrogel beads, a device can perform rapid, simple, and cheap multiplexed protein detection and quantification. Due to the low instrumental complexity required for electrical measurements any potential device using these particles can be highly portable and cost effective to produce.

In an embodiment, provided herein is a multiplexible particle system for use with an electronic detector to detect a plurality of distinct targets. The multiplexible particle system may comprise a plurality of monodisperse polymer particle populations, each population having a unique electrical parameter during flow through a spatially confined electric field in the electronic detector for multiplexed detection. The unique electrical parameter has an electrical parameter distribution for each polymer particle population flowing through a spatially confined electrical field of the electronic detector, and the distribution in a given polymer particle population is sufficiently narrow to minimize overlap with any other polymer particle population to achieve the multiplexed detection.

Each of the polymer particle populations can be described in terms of an average density and an average diameter. In an embodiment, those average diameters may be further described in terms of a difference between any two populations. Preferably, the average density difference and/or the average diameter difference between any populations are selected to be low enough such that during use substantially equivalent flow properties during flow in a suspending solution. This ensures that each population has similar fundamental flow properties to minimize risk of a different flow property (e.g., settling, contacting an element in a capture chamber, etc.) in a population adversely impacting detection. For example, the average density difference may be less than 30%, the average diameter difference may be less than 20%.

Also provided herein are multiplexible particle systems, including systems comprising: a plurality of monodisperse polymer particle populations, each population having a unique electrical parameter for multiplexed detection by flow through a spatially confined electric field; wherein each polymer particle population has an average density that is within 30% of any other polymer particle population and is within 30% of a suspending solution density, wherein during use with the spatially confined electrical field the polymer particle populations are suspended in the suspending solution. The suspending solution typically comprises an electrolyte solution that conducts electrical current through the spatially confined electrical field, such that the presence of a particle in the spatially confined electric field is detectable.

The plurality of polymer particle populations may be described as each having an average diameter, and the maximum average diameter difference between any two populations is less than or equal to 30%, 20%, 10% or between 0 and 30%. In other words, the population having a maximum average diameter (Dₘₐₓ) and the population having a minimum average diameter (Dₘᵢₙ) are described as having (Dₘₐₓ−Dₘᵢₙ)/Dₘₐₓ≤0.3; 0.2; less than 0.1; or between 0 and 0.3.

The polymer particles may comprise cross-linked monomers and/or polymers that form a meshwork scaffold having functional groups corresponding to conjugation sites. In this manner, the polymer particles may be functionalized, such as for specific target binding.

At least one polymer particle population may comprise one or more solid particles embedded in a hydrogel. Any of the polymer particles may be described as hydrogel particles. Any of the polymer particles may be described as non-hydrogel particles. Accordingly, the polymer particle population may comprise both a hydrogel particle population and a non-hydrogel particle population. At least one solid particle may be a magnetic particle. The at least one solid particle may be selected to influence the electrical parameter, particularly for solid particles having a different electrical parameter than the surrounding polymer particle. This may be achieved by having solid particles formed from an electrically insulative material. If it is of interest to minimize change in polymer particle density, the solid particle may be selected to have a density that is substantially matched to the polymer density, such as within 30%, 20% or 10%.

The multiplexible particle system may have at least one polymer particle population comprising hydrogel particles. The hydrogel particles may be described in terms of the amount of water in the particle, such as comprising at least 20%, 40%, 60% or 80% by weight water. Generally, the amount of water may be used to match the particles to a suspending fluid density. For a suspending solution density approaching that of water, the hydrogel particles may have a higher weight content.

The multiplexible particle system may be characterized as comprising microparticles, such as at least one of the particle populations having an average diameter that is less than 1 mm.

The multiplexible particle system may be characterized as having each of the plurality of polymer particle populations having an average diameter that is less than 500 μm, including between about 10 μm and 20 μm.

The multiplexible particle system is compatible with a range of solid particles. For example, the solid particles may have an average diameter that is greater than an average pore size in the polymer particle; and/or are covalently linked to the polymer particle. The solid particles may, in turn, have functionalized surfaces to facilitate specific binding to a target, such as a target cell, protein, protein fragment, polynucleotide, virus, etc. The solid particles may be nanoparticles, having an average diameter less than 1 μm. The solid particles may comprise a mixture of solid particles, including microparticles (ranging in size between 1 μm and 1000 μm) and nanoparticles (ranging in size between 1 nm and 1000 nm).

The spatially confined electric field may correspond to a microchannel of a coulter counter.

The suspending solution may comprise an aqueous electrolyte solution having a density of between 1 g/cm³ to 1.9 g/cm³.

The unique electrical parameter may correspond to a measured electrical impedance during flow through the spatially confined electrical field, wherein each polymer particle population is characterized by a coefficient of variation of the measured electrical impedance. Generally, smaller variation is better for higher level of multiplexibility. Of course, the invention is compatible with much larger variation, depending on the application of interest, including for a smaller level of multiplexibility. As but one example, the coefficient of variation of the measured electrical impedance may be less than 15%. Such a quantitative definition, in certain embodiments, may be characterized as satisfying a sufficiently narrow distribution of the population's electrical parameter.

The polymer particle populations may be selected to each have an electrical parameter with a mean value and a standard deviation such that during use the system identifies a polymer particle to its corresponding population with an error rate that is less than 10%. In this context, conceptually the distribution of the polymer particle populations are such that, for a given particle detection, there is less than 10% likelihood that the particle is misclassified with respect to its population. This error, in turn, relates to the relative overlap between “electrically” adjacent populations, so that for lower error rates, there is less overlap between the “tails” of the adjacent populations.

The unique electrical parameter is selected by adjusting one or more of: polymer composition; polymer size; polymer density; presence or absence of a solid particle within the polymer; volume fraction of solid particles within the polymer; functional groups in the polymer that affect polymer hydration status; or organic and/or inorganic moieties attached to the polymer particles.

The multiplexible particle system is compatible with a range of materials, such as a polymer particle formed from a material selected from the group consisting of: polyacrylamide; poly(N-isopropylacrylamide); alginate; agarose; poly(ethyleneglycol)diacrylate; polyacrylate; polyvinyl alcohol; copolymers having an abundance of hydrophilic groups; and a mixture of any two or more of the above materials.

The multiplexible particle system may comprise between 2 and 100 distinct populations, including between 2 and 10; 2 and 5; 2 and 4; and 2 and 3.

The multiplexible particle system may further comprise a tag connected to and/or embedded in at least one polymer particle population. The tag may be selected from the group consisting of one or more of: an optical label; a magnetic particle; a receptor molecule; a target molecule; and groups that are orthogonally reactive.

Any of the systems provided herein may be described as a kit comprising a plurality of the polymer particle populations. The populations may be tailored to a specific application of interest, including an instrument that will be used to detect the populations in the application of interest. For example, relevant particle sizes and properties may be provided matched to an aperture size and desired flow-rate. For applications having a relatively high error rate tolerance, more overlapping electrical parameter populations may be provided, thereby increasing the number of distinct populations. Written instructions may be provided with the kit, such as providing compatibility to coulter counter apertures, flow rates, suspending media, target specificity, and specific operating instructions, including for a method using the kit. One or more of an appropriate suspending media, magnets, capture chambers, may be provided in the kit. Additional multiplexibility is available by combining with other distinct-signal producing technologies, such as optical multiplexing, electrochemical multiplexing, and the like. Accordingly, the kit may contain corresponding additional reagents and materials to achieve this additional multiplexing, including tags (dyes, labeled beads, etc.) and corresponding instructions to achieve the additional multiplexing.

Also provided herein are methods of making any of the electrically-multiplexible particle populations described herein.

A method of making electrically-multiplexible particle population may comprise the steps of: flowing a plurality of unique pre-polymer solutions and an immiscible fluid through a drop-forming junction, including a flow-focusing junction, to form a plurality of liquid droplets suspended in the immiscible fluid; providing a surfactant to the plurality of liquid droplets suspended in the immiscible fluid; polymerizing the contents of the plurality of monodisperse liquid droplets to make the plurality of electrically-distinct polymer particle populations; breaking and opening the plurality of polymerized droplets to disperse a plurality of monodisperse polymer particle populations into an aqueous solution; and wherein each population of particles has a distinct electrical impedance signature when flowing through a spatially confined electrical field.

The method may further comprise the step of selecting each of the plurality of pre-polymer solutions and/or further processing at least one polymerized polymer particle population; to generate the unique electrical impedance signature during flow through the spatially confined electrical field, wherein each polymer particle population is characterized by a coefficient of variation of the measured electrical impedance that is less than 15%.

The method may further comprise the step of selecting polymer particle populations to each have a standard deviation during electrical detection, so that a polymer particle population electrically distinguishable error rate is less than 10%.

The selecting step comprises varying one or more of: polymer composition; polymer size; polymer density; presence of one or more solid particles within the polymer; volume fraction of solid particles within the polymer; a concentration of polymerizable monomers or prepolymers; a flow rate of the pre-polymer solution to adjust a particle size; a flow rate of immiscible fluid to adjust particle size; a dimension of the flow-focusing junction; a polymerization process, including a polymerization process that comprises radical initiation; optical initiation; thermal initiation; chemical initiation; or electrochemical initiation; and a concentration of a polymerization initiator.

The method may further comprise the step of adjusting a functional group density (e.g., number of functional groups per unit area) on the polymeric particles, by altering the ratio of polymer components containing functional groups to the polymer components without functional groups, in order to tune the sensitivity detection range. Functional groups are chemical sites that enable attachment of specific targeting groups. If the functional group density is higher, the polymeric particles have greater binding to the target at lower concentration, and vice versa, allowing for adjustment of the detection sensitivity range depending on the application of interest. In this manner, the functional group density is tuned to the application of interest, with higher group density for higher sensitivity.

The method may further comprise the step of adding a separation tag to at least one of the pre-polymer solutions. Exemplary separation tags may comprise: a magnetic particle, such as an iron oxide particle; a chemical or biological functional group, such as an antibody, polynucleotide; or chemical moiety.

The method may further comprise the step of conjugating each population to a unique target-detecting molecule. The target-detecting molecule may be selected from the group consisting of: an antibody that binds a protein biomarker; an oligonucleotide that specifically binds a target sequence; a polypeptide that specifically binds a target protein sequence; an aptamer that specifically binds to a target protein; a protein that specifically binds to a target protein; and chemical moieties that are orthogonally reactive.

The method may make any number of particle populations, such as wherein between 2 and 100 particle populations, and any sub-ranges thereof.

The method may be used in an application selected from the group consisting of: electrical counting; electrical barcoding; electrical sorting; and a degradation assay.

Provided herein are various applications that use any of the multiplexible particle systems described herein.

For example, provided is a method of detecting and/or quantifying a target molecule, the method comprising the steps of: providing a multiplexible polymer particle system; conjugating each polymer particle population with a unique target detection molecule; contacting the plurality of polymer particle populations with a sample comprising a target molecule that specifically binds to a specific target detection material, thereby binding the target molecule to the polymer particle; flowing the polymer particle populations past an entrance detector; counting the number of polymer particles in each population that pass the first detector; selectively capturing polymer particles in a capture chamber; flowing the polymer particles not captured in the capture chamber past an exit detector; counting the number of polymer particles in each population that pass the exit detector; determining the difference in flowing polymer particles past the entrance and exit detector, thereby detecting the target molecule.

The method may further utilize a sandwich-type structure in the presence of the target molecules, facilitating the capture of all polymeric particles using a single “common” capture mechanism. For example, if the capture chamber displays streptavidin for capture, biotinylated secondary antibodies or biotinylated nucleotides will need to bind to the target molecules captured on the polymer particles so that the entire microparticle complex is captured in the capture chamber. The capture chamber may further comprise an array of capture elements to facilitate binding of appropriate target polymer bead population(s). For example, the capture elements may be relief features extending out of a capture chamber surface into a flowing suspending media in which the particle populations are suspended. Examples of relief features include posts, pillars, membrane surfaces and the like. For example, the relief features may comprise posts that are cylindrically-shaped with a circular cross-section. The posts may be an array of microposts having dimensions that are micrometer-sized (e.g., at least one dimensnion less than 1 mm, less than 100 μm, and less than 50 μm).

There can be a multiplex method capable in a single run of detecting two or more target molecules, the method further comprising the step of: identifying the polymer particle population of a polymer particle that passes the detectors by measuring the unique electrical parameter as the particle flows past the detector.

The method may further comprise determining a concentration of target molecules in the sample by: obtaining a calibration curve; measuring the amount of a captured particle population; and determining from the amount of captured particle population and the calibration curve the concentration of target molecules in the sample.

The method may have a target molecule selected from the group consisting of: a DNA sequence; an RNA sequence; an amino acid sequence; a cell surface protein; a protein biomarker from a biological sample; and chemical moieties that are orthogonally reactive to functional groups of the polymer particles.

Any of the application methods described herein may be implemented in a point-of-care device, including a point-of-care device to identify a disease condition.

The method may further comprise the step of magnetically capturing particle populations having a magnetizable particle in the polymer. The method may further comprise the step of releasing the magnetically captured particle populations.

Also provided herein are applications related to electrical barcoding, including a method of encoding a fluid material identity, the method comprising the steps of: introducing a multiplexible particle system described herein to a fluid material, wherein the ratio or presence of each polymer particle population is known, thereby encoding the fluid material identity.

Also provided is a method of identifying an encoded fluid material, including by: obtaining a fluid sample of the encoded fluid material; flowing the fluid sample past a confined electric field; and determining a ratio or presence of each of the particle populations in the fluid sample, thereby identifying the encoded fluid material. The method may further comprise the step of encoding the fluid material identity by: introducing any of the multiplexible particle systems provided herein to a fluid material, wherein the ratio of each polymer particle population is known, thereby encoding the fluid material identity

The method is compatible with range of fluid sample volumes, including a fluid sample volume that is between 1 μL and 10 mL, including between 1 μL and 1 mL.

Any of the methods provided herein may have a confined electric field that is produced by a coulter counter.

Further provided is a method of electrically barcoding a material, the method comprising: encapsulating the material inside an electrically distinct polymer particle, including any of the polymer particle populations described herein. The method may further comprise the steps of: flowing the encapsulated material inside the electrically distinct polymer particle through a confined electric field; and detecting an electrical parameter, including an electrical impedance, as the encapsulated material inside the electrically distinct polymer particle flows through the confined electric field, thereby identifying the encapsulated material.

The material may correspond to: a biological cell; a polynucleotide; a polypeptide; a protein; a virus; a drug; or a chemical.

Also provided herein are methods of electrical sorting. For example, provided is a method of electrically sorting a sample including any of the polymer particle populations described herein, including by: actuating a controller based on a measured electronic parameter as the polymer particle passes through a confined electric field to collect a desired polymer particle population.

The controller may be selected from the group consisting of: a valve to redirect flow; an electrode to generate a dielectrophoretic force on the polymer particle population; an acoustic generator to redirect flow; and an electromagnet to generate a magnetic force on the polymer particle population.

Also provided herein are highly multiplexed assays, wherein degradable components are utilized to further increase multiplexibility beyond the n distinct particle populations.

A highly multiplexed detection method may comprise the steps of: providing a plurality of polymer particle populations, wherein each population has an electrical signature and at least one of the populations have a degradation parameter; first flowing the plurality of polymer particle populations through a confined electric field and measuring the electrical signature of the polymer particles passing the confined electric field; applying a degradation stimulus to the polymer particles that have passed the confined electric field, wherein the degradation stimulus is targeted to at least one degradation parameter, thereby degrading polymer particle populations having the targeted degradation parameter to generate a degraded plurality of polymer particle populations; and second flowing the degraded plurality of polymer particle populations through a confined electric field and measuring the electrical signature of the polymer particles passing the confined electric field.

The method is compatible with a range of stimuli, including a degradation stimulus selected from the group consisting of: a chemical stimulus; a biological stimulus; a temperature stimulus; a pH stimulus; and an electromagnetic stimulus;

The highly multiplexed detection may be characterized by: detection of N particle populations for n distinct electrical signatures, where: n<N≥2n.

The confined electric field in the first and second flowing steps may be the same (e.g., a feedback loop) or different (e.g., two distinct counters) confined electric fields.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A. Multiple polymer particles (e.g., hydrogel beads) that are similar in size but produce distinct electrical signals. The lines through the polymer particles denote current (I) flowing through the hydrogel beads. Any one or more of bead size, density, and solid volume can be independently varied to achieve a distinct electrical parameter, such as impedance, for each of the four illustrated polymer particle populations. FIG. 1B. General chemical structure of a polymer particle, such as a hydrogel network. Monomers and crosslinkers form a meshwork scaffold, while specific functional groups provide sites for conjugation to biomolecules.

FIG. 2A shows droplet generation in the dripping regime. The gel forming solution is encapsulated into monodisperse droplets immersed in fluorinated oil. The channel widens rapidly after droplet formation to minimize the shear stress on the droplets. FIG. 2B shows microscope image of the droplets after polymerization has occurred during heating. The droplet size remains monodisperse and merging is minimal. FIG. 2C shows microscope image of a single population of the hydrogel beads after absorbing water and swelling to their maximal size. FIGS. 2D-2G show phase contrast images of the four synthesized bead populations (magnetic hydrogel beads or MBH); FIG. 2D MHB1, FIG. 2E MHB2, FIG. 2F MHB3, FIG. 2G MHB4.

FIG. 3A shows the impedance of the coulter counter is monitored as voltage. This short span of time shows the recorded voltage as 3 beads pass the detector. The measured voltage indicates that the first bead belongs to population 4, while the second bead belongs to population 2, etc. The measured impedance signals are sufficient to identify each bead. FIG. 3B shows the histograms of bead events collected by voltage. Three distinct populations are present and sufficiently spaced so that the electrical signal is sufficient to distinguish each bead. The region at low impedance results from electrical noise and is excluded from bead counting analysis. FIG. 3C shows flow cytometry measurements of the fluorescent intensity each population of beads following conjugation at different biotin concentrations. Following conjugation to biotin, each bead population was incubated with an excess of fluorescently-labeled streptavidin which binds to biotin converting the successful conjugation events into an optical signal that is detected by the flow cytometer. These results show that conjugation to the beads occurred successfully and that the fluorescent signal is proportional to the biotin concentration during conjugation . FIG. 3D shows magnetization plots of MHBs at 300 K and at 5 K (inset). These magnetization plots confirm that the produced hydrogels are magnetic and have successfully incorporated the magnetic nanoparticles inside. FIG. 3E shows confocal images of MHB1 conjugated to biotin and labeled with fluorescent streptavidin. FIG. 3F shows 3-D reconstruction of fluorescently labeled MHB1. FIGS. 3G-3I are electrical impedance traces of MHB1, MHB2 and MHB4, respectively, passing the passing the Coulter counting electrodes at the entrance to the device. The biphasic signal helps to resolve particles from the noise.

FIG. 4. Design of a differential counting device that can be used with a multiplexible particle system. Beads are counted at the entrance and exit counters. Each polymer particle population is captured in proportion to the concentration of the protein target using a sandwich immunoassay, such as with biotin-streptavidin capture. The capture chamber provides many streptavidin coated surfaces that capture polymer particles that have been tagged with biotin as a result of the protein binding events on the polymer particle surface.

FIG. 5A. shows percent capture plots obtained using a differential counting device. Particles are counted at the entrance and exit of a streptavidin functionalized capture chamber. Each population of MHBs are captured selectively using the biotin streptavidin interaction. This type of calibration curve can be used to quantify target in terms of percent particle capture. FIGS. 5B-D. The particles are counted as they enter and exit to produce the % capture, for each of biotin concentration of 0, 4 and 20 μg/m L, respectively. As the concentration of biotin (biomolecule) increases, larger fraction of the particles are captured.

FIG. 6. Microscope Image of the hydrogel beads captured to a post inside the capture chamber using biotin-streptavidin interactions. This image confirms that the beads are being captured as described.

FIG. 7 is a scheme for detection of polynucleotide sequences using MHBs.

FIG. 8 is a schematic summary of an electrical barcoding application, wherein each polymer particle population corresponds to a parameter that encodes or identifies a material, illustrated as sample 1, 2 and 3. A portion of the material can be removed and flowed through a spatially confined electric field to detect presence, absence and/or relative amount of the polymer particle populations.

FIG. 9 is a schematic illustration of electrical sorting using any of the multiplex systems described herein. Relevant material can be contained in the polymer particles, with each unique material corresponding to a unique polymer particle population. A mixed sample can be sorted by electrically identifying the populations and correspondingly sorting by population to achieve sorted samples from a mixed sample.

FIG. 10 schematically illustrates a degradation step to achieve additional multiplexing. The first three panel steps are similar to FIG. 4. Further multiplexing is achieved by degrading specific polymer particle population(s), such as populations having a composition that are degradationally sensitive to a physical parameter, such as pH, temperature, turbidity, chemical attack, electromagnetic attack, etc.) with subsequent counting, thereby achieving higher multiplexing, even for populations having similar or overlapping electrical signatures during detection.

FIG. 11A shows a chemical structure of an exemplary polyacrylamide-based hydrogel. Acryloyl-β-alanine (ABA) monomers incorporate carboxyl groups into the network for further conjugation to biomolecules. FIG. 11B shows four synthesized MHBs that are similar in size yet produce distinct electrical signals. Lines through the MHBs denote the current (I) at the coulter counter. Since the hydrogel beads are filled with a conductive buffer solution some current can flow through the hydrogels. Larger beads or more dense hydrogels both result in increased electrical impedance. Black circles denote magnetic nanoparticles. The impedance signal produced by each bead is proportional to the amount of hydrogel material it contains

FIG. 12A shows scanning electron microscope images of a dried sample of MHB2. The shape of the dried beads can be seen. FIG. 12B shows transmission electron microscope images of a dried sample of MHB2. Speckles can be seen dispersed throughout each gel due to the incorporated magnetic nanoparticles.

FIG. 13A is hydrogels produced using heat induced polymerization method. FIG. 13B illustrates hydrogels produced using TEMED diffusion into droplets to accelerate polymerization. The resulting gels swell to different sizes in buffer producing a polydisperse sample.

FIG. 14 is a schematic of one embodiment of a multiplexed bead counting device. Beads are counted as they flow past the aperture of the entrance Coulter counter (labelled “Entrance Counter” and shown in bottom left image). Beads are designed to produce distinct impedance signatures for each bead population to enable multiplexing. After counting, the beads are captured as they flow through an array of pillars (labelled “Capture Image” and shown in bottom middle image perspective view of the pillars and bottom right image is a top view of the pillars) inside the capture chamber. The beads pass a second Coulter counter (“Exit Counter”) as they exit the device. The capture percentage is measured and correlated to the concentration of the target analyte.

FIG. 15 illustrates a droplet microfluidic beadmaking device. Junction dimensions are approximately 7 μm×7 μm with a height of 10 μm. Stable drop-making is achieved in the dripping regime. The device widens rapidly downstream of the junction to reduce the shear force on the droplets.

FIGS. 16A-16F are photomicrographs of drop-maker and resultant monodisperse bead population. FIG. 16A shows the gel forming solution is encapsulated into monodisperse droplets in fluorocarbon oil. FIG. 16B: Image of the droplet emulsion after heat induced polymerization. FIG. 16C: Image of the same population of beads after swelling in PBS. FIG. 16D: Phase contrast images of the four bead populations. Scale bar is 20 μm. FIG. 16E: A suspension of stained cells was mixed with similarly sized magnetic hydrogel beads. The cells and beads can be easily distinguished by fluorescence. FIG. 16F: Following magnetic separation, the cells are removed.

FIGS. 17A-17B SQUID Magnetometer plots for each bead type collected at 300 K (FIG. 17A) and at 5 K (FIG. 17B). The observed behavior confirms that the hydrogel beads are rendered magnetic by the incorporation of the nanoparticles.

FIGS. 18A-18D are histograms of the different bead populations showing the counts binned by voltage for MHB1, MHB2, MHB3 and MHB4, respectively. The impedance signals of MHB2 and MHB3 are overlapped since the effects of the increase in density are counteracted by the reduction in bead volume.

FIG. 19A is a comparison of nonspecific capture between three electrically distinct solid magnetic beads and MHBs at the same flow rate. Each type of bead was captured by separate chambers. FIG. 19B is a plot of fluorescent intensity of MHBs and solid magnetic beads after biotin conjugation. The MHBs show greater functional group density.

FIG. 20 COMSOL simulations of the magnitude of the surface velocity inside the capture chamber; streamlines are also indicated. The green (right image) and red particles (left image) have diameters of 8 and 13 μm respectively. These results show location of low velocity regions present behind the pillars. These regions have the highest likelihood of nonspecific capture. Since beads of different sizes experience the flow profile differently, size variations impact the rates of nonspecific capture.

FIG. 21 COMSOL simulations of the shear stress inside the capture chamber of the device at different flow rates. Redder areas indicate higher shear stress, while bluer regions have lower shear stress. As the flow rate increases the shear stress at the pillars increases decreasing the likelihood of bead capture.

FIG. 22 summarizes flow cytometry measurements comparing the efficiency of surface functionalization between MHBs and commercially available latex beads. Since the MHBs have higher surface functional group density, they produce a larger fluorescent response relative to the concentration of biotin during functionalization.

FIG. 23A Inset: 3-D reconstruction of a fluorescently labeled MHB. Orthographic projection of the same bead reveals the distribution of fluorescence on the surface of the bead. FIG. 23B Fluorescent intensity each population of beads following conjugation at different biotin concentrations, measured by flow cytometry. FIG. 23C Percent capture for MHB1, 2 and 4 at different levels of accessible biotin. FIGS. 23D-23E Bead counting histograms obtained from a mixed sample containing MHB1, MHB2, and MHB4, one population is conjugated to biotin: FIG. 23D MHB1 or FIG. 23E MHB2. The overlaid exit counts show a significant drop for the biotin conjugated bead as it is captured.

FIGS. 24A-24B are confocal images of MHB2 and MHB4, respectively, after conjugation to biotin and fluorescent labeling by streptavidin binding.

FIG. 25 is an image showing various MHBs captured to pillars inside a capture chamber of the device.

FIG. 26A Multiplexed protein measurement showing specific capture of IL-6. FIG. 26B Multiplexed DNA measurement showing specific capture of TP53 fragments. In both cases MHB1, MHB2, and MHB4 are flowed simultaneously through the same chamber.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

“Multiplexible” refers to the ability to distinguish a plurality of targets/events/states in a single experimental run. For example, for n electrically-distinguishable polymer particle populations, n different targets can be detected. “Highly” multiplexible refers to the ability to detect N different targets using n electrically distinguishable polymer particle populations, where N>n. This is achieved by adding further distinguishing capability to the populations, such as with a tag, functional group, magnetic material, degradation capability and/or the like.

“Sufficiently narrow”, as further described below, relates to a statistical measure of a distribution or dispersion about a mean, including a standard deviation, coefficient of variation, variance, and reflects a population is “monodisperse”. The systems and methods provided herein are compatible with any of a range of magnitudes of distributions, so long as the populations are capable of being reliably distinguished from one another so that reliable multiplexible identification is achieved. Accordingly, the degree or specific quantitative measure of sufficiently narrow depends, at least in part, on the application of interest. If only two populations or two targets are being measured, the term sufficiently narrow reflects that the dispersion in each population can be higher than if more than two populations or more than two targets are being measured. So long as the detector is capable of electrically identifying a passing particle as belonging to a single population. The larger the distribution, and the closer together the means, the higher the likelihood of an “overlap” with attendant uncertainty as to which population the particle belongs to. Sufficiently narrow may refer to a distribution of measured impedance for each particle of a population passing the confined electric field generated by an electrical detector, including a Coulter Counter® apparatus.

In certain contexts when referring to the instant multiplexible systems, the term “particle” and “bead” are used interchangeably. A particle or bead refers generally to the spherically shaped polymers that can be used for the instant multiplexible applications. As is apparent, particle in other contexts may refer to a solid material contained within the polymer particle, that is used to modify an electrical signature or for further multiplexing via a different, “orthogonal” signal, such as a magnetic force, an electric field, a binding interaction, or the like.

“Monodisperse” refers to a relatively uniform population (e.g., the population has a “sufficiently narrow” dispersion) in terms of an electrical parameter associated with the population, reflecting the invention is compatible and tolerates some degree of non-uniformity. A precise quantification of the uniformity depends, at least in part, on the end application, detector sensitivity, and various parameters related thereto, such as the number of different “events” that are desirably detected, e.g., degree of multiplexing. An individual population is typically described as having a Gaussian or “normal” distribution. Accordingly, the uniformity may be characterized by the standard deviation (SD or σ) relative to a mean, as the (average±σ) corresponds to about 68% of the total number of particles in the monodisperse polymer particle population for a normally distributed population. Accordingly, the populations may be described in terms of a standard deviation of the mean (SEM), wherein SEM=σ/n^{1/2}; the relative standard error is σ/average, and any of the populations provided herein may be less than 20%, less than 15%, or less than 10%. The systems and methods may be characterized as providing a desired confidence limit when electrically detected by an instrument, such as a confidence of at least 90%, or at least 95%.

“Electrical parameter” refers to a parameter that can be detected by an electrical-measuring instrument, specifically a detector that can measure changes in the electric field as a particle passes a spatially confined electric field. “Spatially confined electric field” refers to a location where the polymer particles traverse and an electric field is present, such that the presence or absence of a particle is determined by a change in the electric field, including as measured by a change in an electrical parameter, such as a voltage, a current, a resistance or impedance. In view of the relationship between voltage, current, resistance and impedance, depending on the electrical detection mechanism implemented by the detector instrument, the terms are intended to be interchangeable. A spatially confined electric field may refer to a channel between two electrodes. Depending on the size of the particles, the channel may be a microchannel. The channel (as well as particle concentration in the suspending electrolyte fluid) is selected to minimize risk of more than one particle passing through the channel at the same time. Accordingly, the channel may have a diameter less than about two times the average diameter of the particle, such as less than two times the average diameter of the smallest sized particle diameter, and/or a diameter that is greater than the average diameter of the largest sized particle. In this manner, particle deformation and clogging are avoided.

“Average density” refers to the average density of a particle population. For composite particles, the density will be an effective density, corresponding to the relative contributions by each of the components of the particle. For example, for solid particles that are embedded in the polymer particle, each of the polymer and the solid particle density contribute to the effective density of the particle. An average density for a population reflects that the instant inventions tolerate some variation in uniformity, so long as the ability to distinguish populations remains. This similarly applies to “average diameter”.

“Hydrogel” refers to a polymeric material having a high water content, such as greater than 30% water by weight, including greater than 80% water by weight. In this aspect, a polymer is broader than a hydrogel, in that a polymer can have a lower water by weight content. Hydrogels, also referred to as a superabsorbent polymer, can absorb very high amounts of water, including up to 99.9% liquid in pure water, with lower amounts in electrolyte solutions. The total water absorbance, or swelling capacity, can be reliably controlled by the composition and degree of cross-linking, as well as use of conjugation sites that have functional groups that may influence hydrophobicity of the hydrogel. For example, lower cross-linking generally corresponds to higher absorbent capacity. More generally, a hydrogel is a hydrophilic network of polymer chains.

“Coulter counter” refers generically to an instrument that measures an electrical parameter of particles passing through an aperture having a confined electric field. See, e.g., U.S. Pat. No. 2,656,508 “Means for Counting Particles Suspended in a Fluid” by Coulter. The aperture may correspond to a microfluidic channel, or a plurality of microchannels, each having an effective cross-section dimension that is less than about 1 mm. As fluid containing beads of the instant invention is drawn through each microchannel, each bead causes a brief change to the electrical resistance of the liquid. The counter detects these changes in electrical resistance. More generally, the change can be characterized as a change in impedance.

“Orthogonally reactive” refers to, for example, functional groups that are specific to one target, while having relatively little specificity to another target. There is some tolerance to non-orthogonality, such as a reaction that is 99% or greater specific to a first target and less than 1% specific to the “orthogonal” non-target.

The invention can be further understood by the following non-limiting examples.

### EXAMPLE 1

### Microfluidic Synthesis of Electrically Distinct Polymer Particle Populations

Referring to the figures, droplet microfluidics is used to produce a multiplexible particle system 10, comprising multiple monodisperse populations of polymer particles (10a 10b 10c and 10d), specifically including hydrogel beads 120.

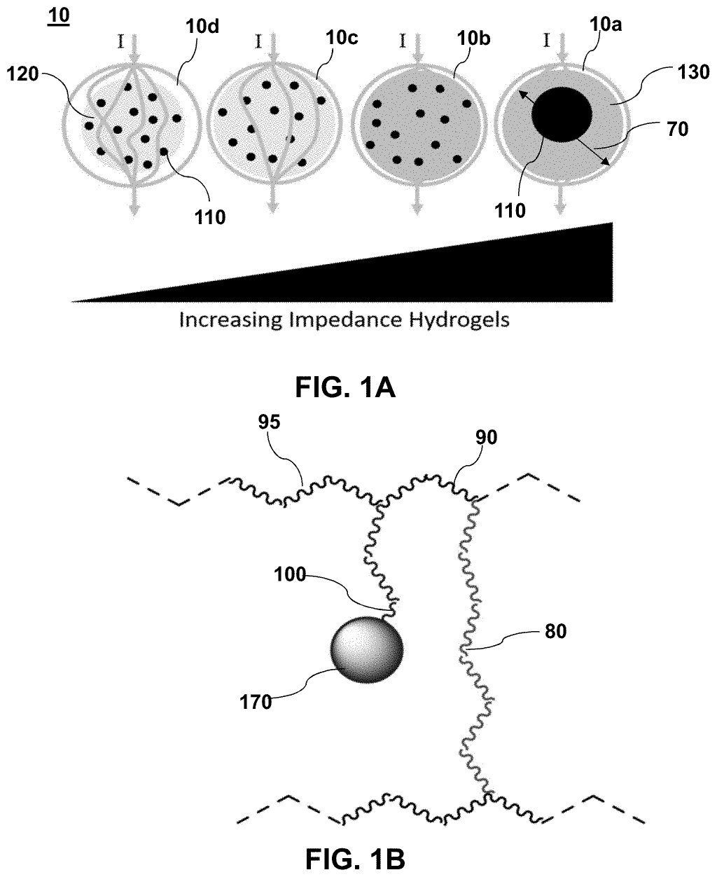

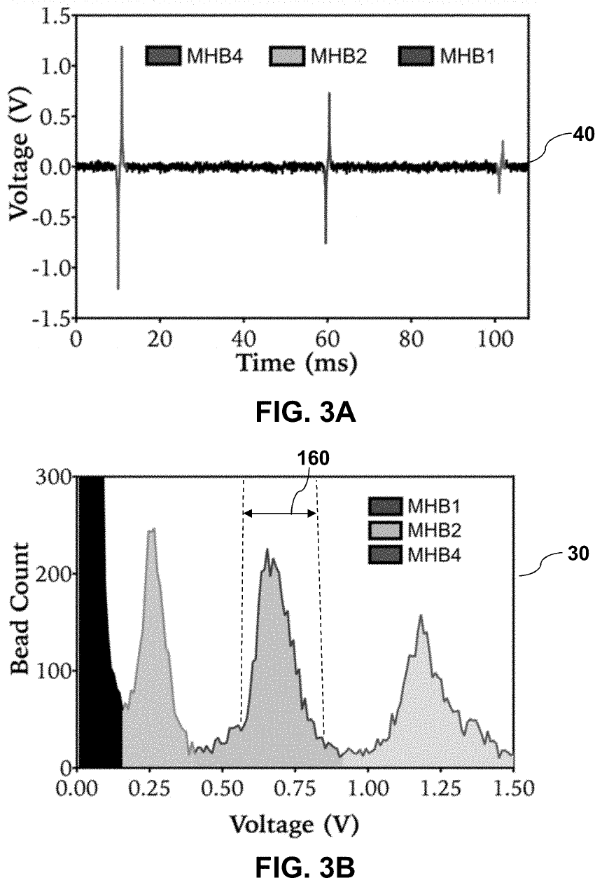

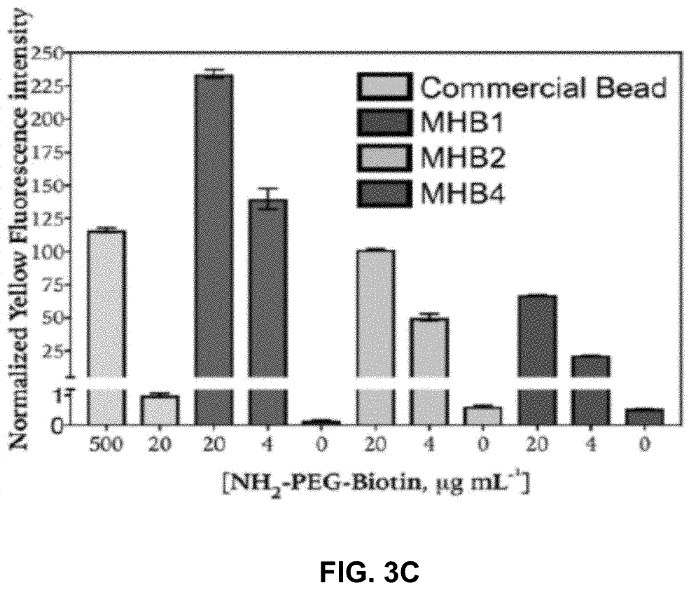

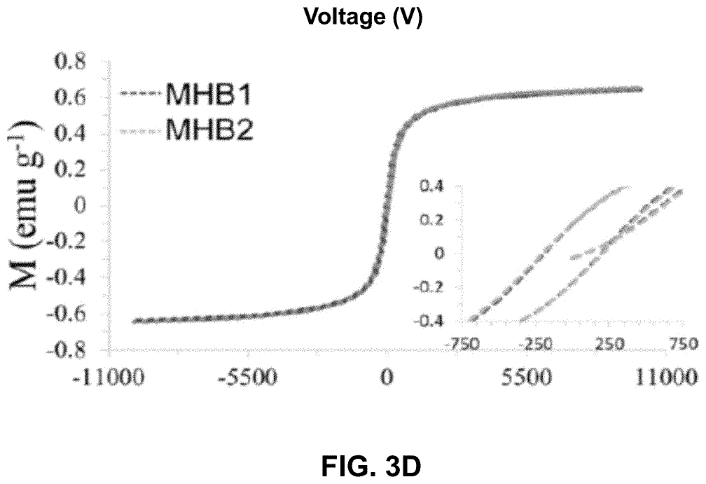

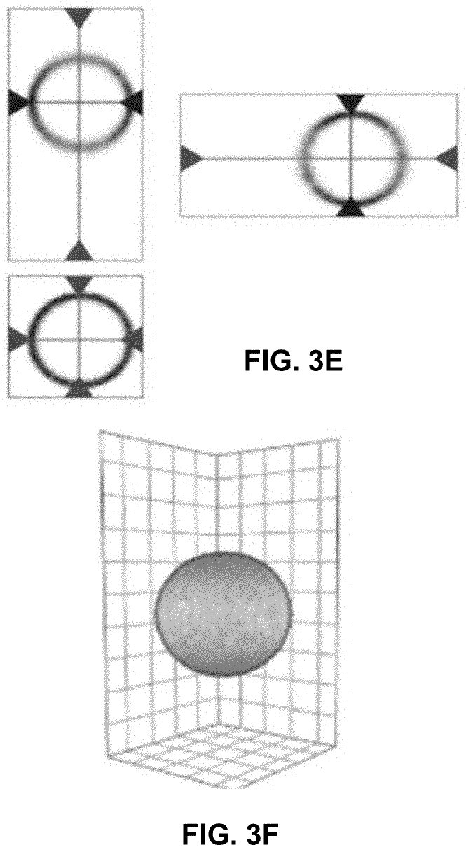

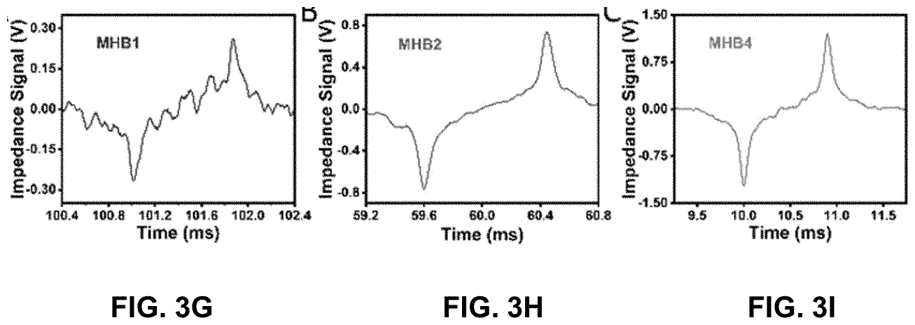

The populations each have distinct electrical impedance signals (or more generically, an electrical parameter 20 measured by the relevant instrument) when flowed past a confined electric field 50, such as produced with a coulter counter instrument 150, for particles suspended in a suspending solution 60, such as an electrolyte solution. Although particles with these engineered properties serve many potential functions, they are particularly useful a multiplexed platform for sensitive and specific biological assays. At a first level of multiplexing, each population of particles is designed to produce distinct impedance signals as they flow through a narrow channel between two electrodes. As the particles occupy the volume between the electrodes a spike in impedance occurs because the polymer particle displaces a portion of the conductive electrolyte medium resisting the flow of current. By designing each population of polymer particles to displace different volumes of electrolyte each population produces distinct impedance signatures (FIG. 1A). Droplet microfluidics allows us to produce multiple populations of polymer particles that are highly monodisperse (low variability within each population). This feature is important for multiplexing because each population will produce only a narrow distribution of impedance signals (see, e.g., FIG. 3B) which makes it possible to unambiguously identify each particle population type with conventional electrical instruments, including instruments based on the Coulter principle.

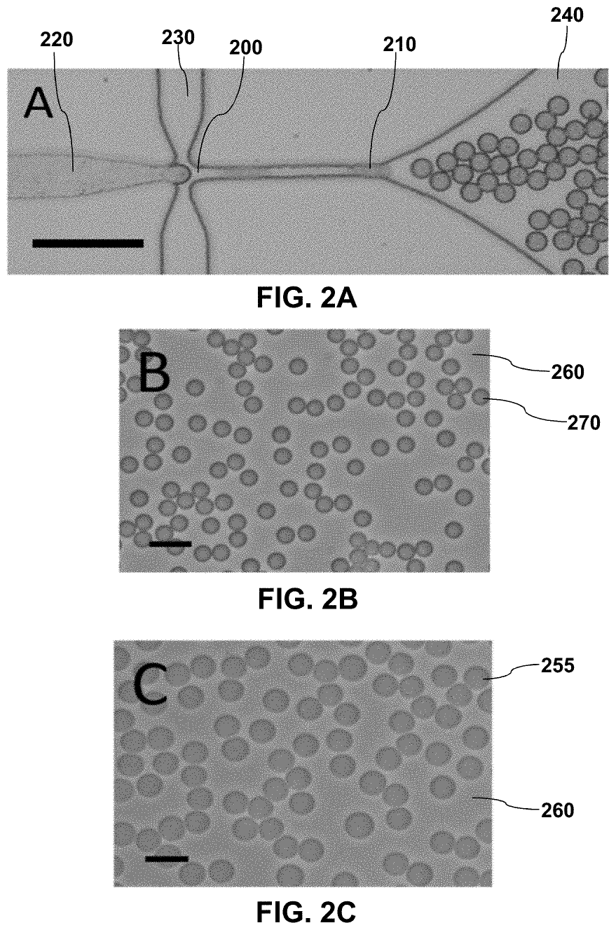

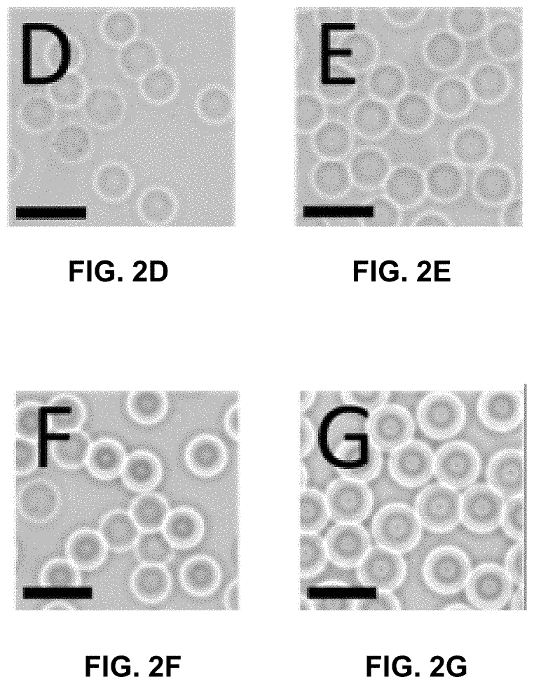

Each population of polymer particles is produced when a water-based solution of monomers and crosslinkers or prepolymers 220 is pushed through a narrow flow focusing junction 200 at the same time as an immiscible fluid 230 (FIG. 2A). This results in the formation of many identically sized spherical water-based solution droplets 210 that are suspended in immiscible fluid, such as oil, FIG. 2B. Surfactants 240 keep the droplets 270 separated as a polymerization reaction occurs. This results in tens of millions of nearly identical polymer spheres 255 per hour suspended in an aqueous solution 260. A population of monodisperse polymeric particles 255 is shown in FIG. 2C. It is possible to produce polymer particles, including hydrogel particles, with distinct electrical signals in multiple ways (FIG. 1A).

Changing the density of the hydrogel corresponds to changes in impedance.

By increasing the concentration of the monomers or prepolymers in the aqueous phase higher density hydrogels are produced which increases electrical impedance.

Changing the size (e.g., diameter) 70 of the polymer particles results in changes in impedance. By adjusting the flow rate, it is possible to produce smaller or larger polymer particle diameters. Similarly, changing the dimensions of the drop-making junction 200 also affects the polymer particle diameter. Larger polymer particles produce higher impedance signals compared to smaller gels of the same composition (compare, e.g., the FIG. 1A population 10d and 10c—increased impedance).

Changing the solid volume of the hydrogel affects the impedance. By incorporating large solid particles 110 a substantial increase in impedance results (compare, e.g., the FIG. 1A population 10b and 10a—increased impedance). The solid particles 110 may be selected to have an average diameter that is greater than an average pore size in the polymer particle 130 (see, e.g., population 10a at top-right of FIG. 1A). In this manner, the solid particles may remain reliably embedded in the polymer interior.

Changing the polymer or hydrogel material may also result in different impedances. Due to differences in the, hydration capacity, ionic strength, flexibility, or other factors different hydrogel materials can produce different electrical signals at a given size and density (compare, e.g., the FIG. 1A population 10c and 10b—increased impedance). It is possible to produce multiple electrically distinct hydrogels by using the principles described above. Additionally, each polymer or hydrogel is synthesized to incorporate useful chemical functional groups that enable facile conjugation to biomolecules using coupling chemistry. FIG. 1B, shows a general chemical structure of the polymer or hydrogel material. A meshwork scaffold 90 is formed by crosslinkers 80 linking monomers and/or polymer backbones 95, with functional groups 100 available to provide conjugation sites to, for example, a tag 170 or the like. The particles can also be made magnetic by incorporating iron oxide particles which enables fast separation and washing.

Multiplexed beads that produce distinct electrical signatures can be used to perform a broad assortment of biological/biochemical tests and functions including multiplexed biomolecule detection, multiplexed cell and biomolecule quantification, electrical barcoding, sorting, and degradation assays.

### EXAMPLE 2

### Detection and Quantification

As a representative example of multiplexed detection and quantification, we can use the multiplexible particle system 10 to perform rapid, cost-effective, multiplexed measurements of 1) cell count, 2) the expression level of a single cell surface protein, and 3) the concentration of multiple protein biomarkers (including 3 or 4) from a blood sample. Importantly the measurement time can be on the order of minutes. This technology has the potential to dramatically improve diagnosis and prognosis at the point-of-care.

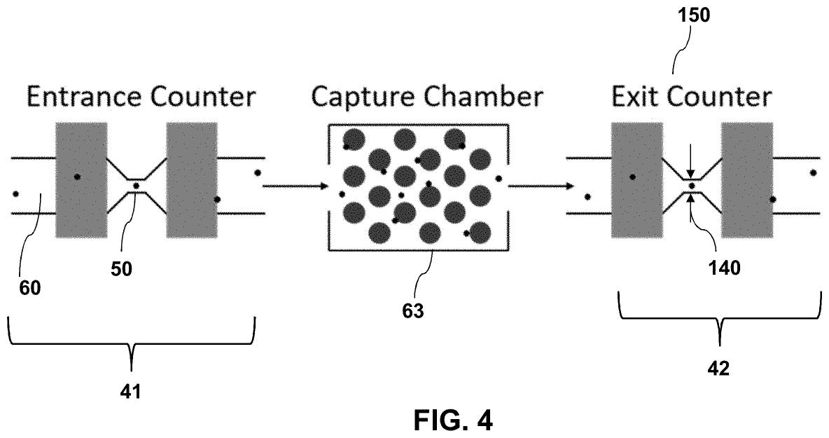

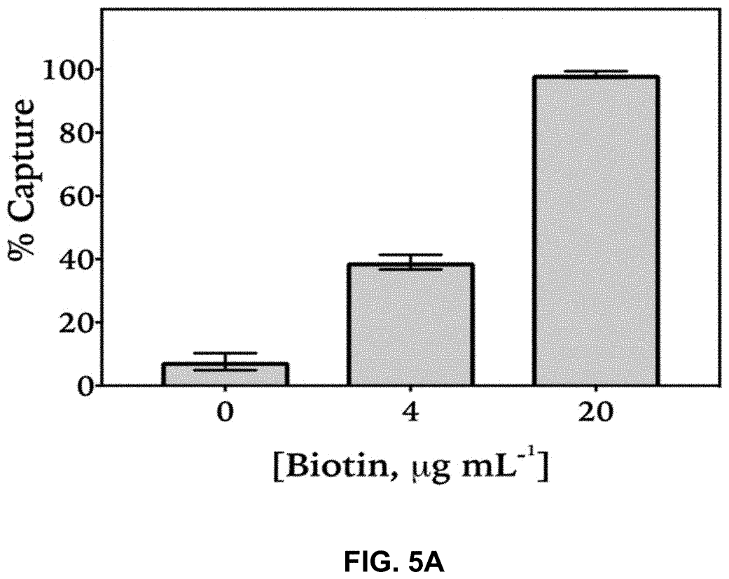

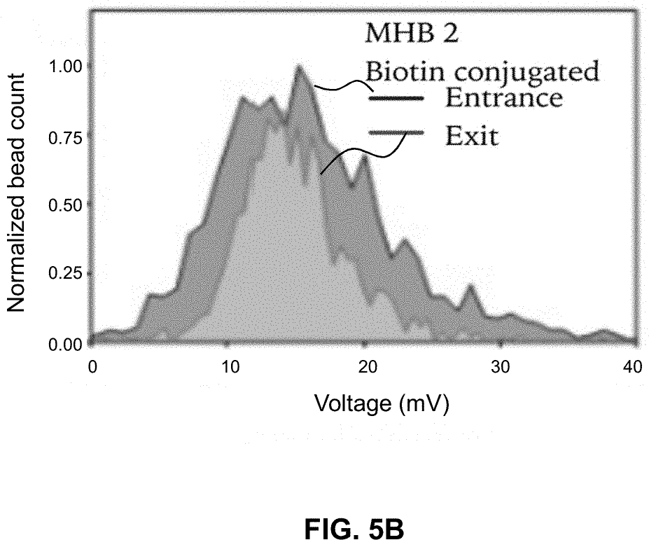

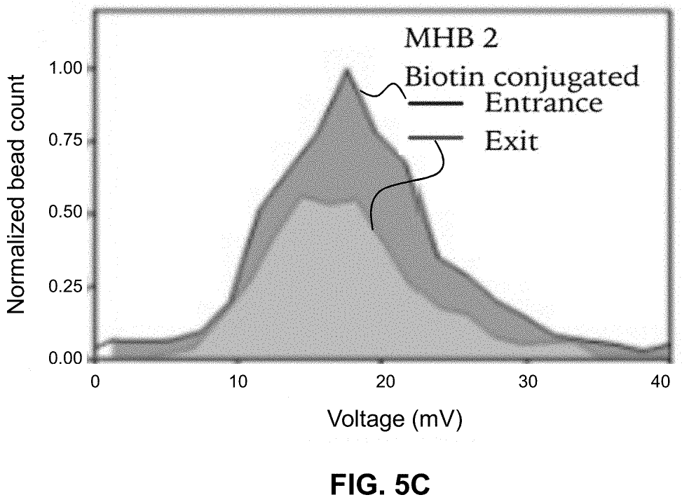

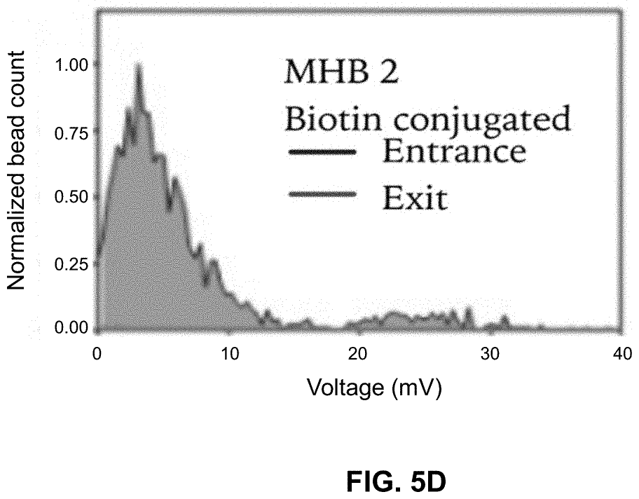

Multiplexed biomarker quantification, such as protein and/or nucleotide sequence quantification, is important for accurate prediction of the onset and progression of diseases as well as early diagnostics since the concentrations of protein biomarkers act as an indicator of disease. Using the inexpensive, sensitive, and rapid measurements enabled by this technology, accelerated diagnosis can be achieved. Early diagnostics helps ensure treatment begins during the earliest stages of disease, thereby dramatically improving outcomes for patients. Following the synthesis of the electrically distinct polymers (hydrogels), each population of beads is conjugated to a specific antibody that targets the protein biomarker. Using established sandwich immunoassay techniques, the beads are tagged with capture functionality in proportion to the concentration of the target biomarkers in the patient sample. The bead solution is then flowed past an initial detector (see entrance counter of FIG. 4) that counts the number of beads of each type using the distinct electrical signals of each bead population (FIGS. 3A-3B). Then the beads are selectively captured inside the capture chamber (FIG. 4) before flowing back to another electrical detector. The exit counter obtains a second measurement of the bead count that gives the percent capture when combined with the initial count. The percent capture can be calibrated to give a measurement of the concentration of the target biomarkers (FIG. 5A).

Since each bead type is selective towards a specific target molecule, the number of simultaneous measurements depends on the number of electrically distinguishable populations. The polymer particles can be magnetic and roughly the same size as cells. This allows for modification of the procedure to obtain a measurement of cell count and the expression level of a cell surface protein using the same device. Although a 10 μm cell produces an impedance signal that overlaps these hydrogel beads, a magnet can be used to retain all the magnetic beads while the non-magnetic cell flow and are counted. Cell surface protein expression can be quantified using immunocapture targeting the protein, and the difference between entrance and exit counts. After the cells are measured, the magnet is removed and the multiplexed bead capture and counting is performed. As such, the polymer particles provided herein enable a cost-effective, rapid, and multiplexed measurement of cell count, the expression of a single surface protein, and multiple protein biomarkers from a single sample in only a few minutes of measurement time.

The systems provided herein are also well-suited for short oligonucleotide detecting using the instant multiplexible particle systems. Each population of polymer particles are conjugated to a unique binding oligo, such as a complementary sequence to a target nucleotide sequence. The multiplexible particle system, with the populations appropriately conjugated to the binding oligos, is mixed with a sample that may or may not contain the target nucleotide sequence (top left panel 700). If the target is present in the sample, the target can be captured by the polymer particle population (e.g., specific binding between target and target sequence 710). In this manner, a plurality of target sequences may be assayed. If there is no target present in the sample, there is no specific binding for that specific polymer particle population. The particle populations can then be mixed with a biotin-tagged probe specific for all target sequences in the assay (730) and counted with a first detector, then passed through a capture chamber, where a biotin-streptavidin interaction captures biotin-tagged particles, and subsequently counted again (see, e.g., FIG. 4) (740). In this manner, multiplex detection of a polynucleotide (DNA or RNA) is provided.

### EXAMPLE 3

### Electrical Barcoding

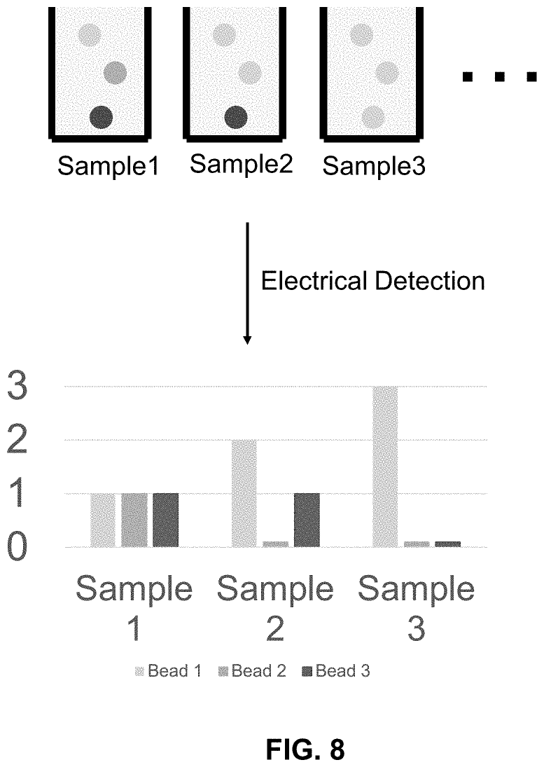

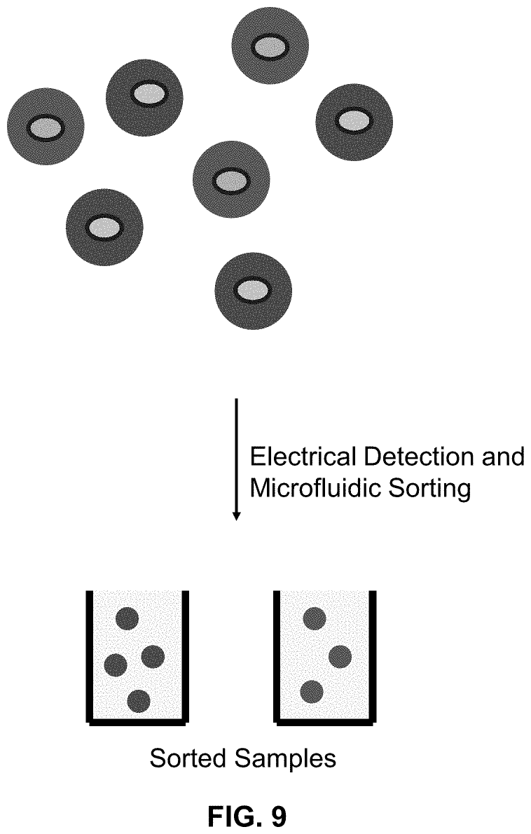

It is often important to encode the identity of samples so that they can be read out at a later time. Electrical barcoding which uses the impedance pattern of the sample to resolve its identity can be performed in two ways using electrically distinct hydrogel beads. Different ratios of each electrically distinct bead can be added to various sample conditions. To read out the identity of each sample a few microliters of solution are removed and measured using a coulter counter. Based on the ratios between each distinct bead signal the sample condition can be unambiguously resolved (FIG. 8). Electrical barcoding can also be performed by encapsulating different samples inside electrically distinct hydrogels (FIG. 9). As an example, certain hydrogel materials are suitable to trap and contain cells as they grow and multiply. In this way different cells can be loaded into electrically distinct hydrogels and allowed to mix and interact. The identity of each sample condition can be resolved by measuring the electrical impedance of each bead.

### EXAMPLE 4

### Electrical Sorting

Along with resolving the identity of a sample, it is similarly useful to separate or enrich the output with a desired set of samples. Electrical barcoding makes it possible to sort beads based on their electrical signals. The impedance measurement is then used to trigger a valve that redirects flow, an electrode that induces a dielectrophoretic force on the bead, an acoustic wave that steers flow, an electromagnet to apply a magnetic force on the bead, or some other method to direct particles towards a specific collection channel. In this way, it is possible to selectively sort beads based on their impedance signals to either enrich or separate out the desired samples (FIG. 8). Typical microfluidic sorting techniques require the optical detection of the sorted particles which is more complex and costly compared to an impedance measurement. Our hydrogel microspheres can be sorted using cheap electrical components.

### EXAMPLE 5

### Degradation Assay

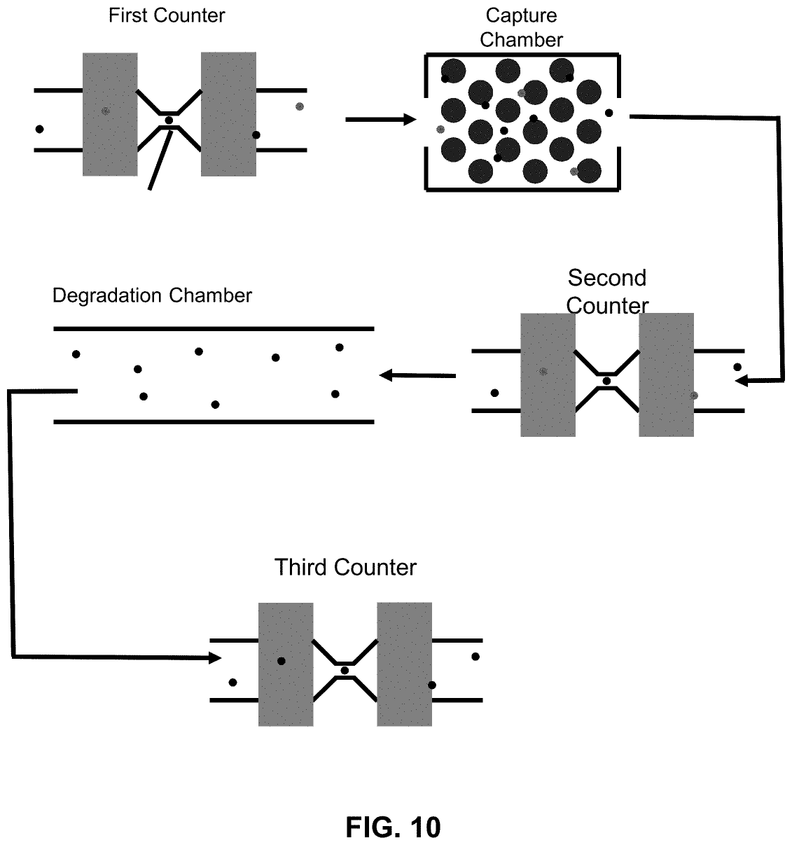

It is also possible to detect and measure the degradation of the hydrogel material as a result of a chemical or biological process. Certain polymers and hydrogel materials breakdown or dissolve in response to heat, pH changes, or other stresses. It is possible to create multiple populations of hydrogels that are first counted, then subjected to a process that results in the breakdown of the gels, and then counted again (FIG. 10). The difference in counts before and after degradation provides an independent measurement of the degradation rate of the different populations. In addition to this, degradation of hydrogels can be used to create more multiplexed electrical signatures. Consider two beads that produce the same impedance but one of them is made of a degradable material. An initial electrical measurement produces a summed count of the two populations. The beads are then subjected to selective degradation followed by a second electrical count. This measurement resolves the fraction of the initial counts belonging to each population making the combined measurement sufficient to electrically distinguish two particles that produce the same impedance using selective degradation. It is possible to extend this idea to double the number of multiplexed measurements as each unique impedance signature is occupied by a degradable and non-degradable bead which can be distinguished using an additional electrical measurement, thus providing “high” multiplexibility.

### EXAMPLE 6

### Systems Using Multiple Magnetic Hydrogel Beads (MHB)

The rapid detection of multiple protein biomarkers from patient samples is crucial to effective diagnosis. We developed a new microfluidic biochip platform integrating a differential coulter counter and an immunocapture chamber that can rapidly quantify a single protein target (or other biomolecules) from patient samples. However, the solid magnetic particles currently employed by the device, restrict the possibility of multiplexing, limiting its applications. New, highly engineered microparticles are needed that feature magnetic properties, surface functionalization, and uniform capture dynamics, while also producing distinct impedance signals. Using droplet microfluidics, we generate multiple monodisperse populations of magnetic, carboxyl-functionalized, polyacrylamide hydrogel beads. Each population of beads is configured to contain increasing amounts of polyacrylamide, resulting in distinct electrical signatures on a coulter counter. Using a differential counting device, we confirm the simultaneous and selective capture of each bead population and demonstrate the dependence of capture efficiency on the biotin loading of the hydrogel. The synthesized beads (particles) illustrated in this example address the challenges associated with multiplexing the bead counting platform and establishes a platform for rapid quantification of multiple biomolecules from a limited patient sample.

The potential to rapidly quantitate a single protein biomarker from human plasma in minutes using coulter counting and immunocapture of a single population of beads has recently been demonstrated.¹ That technique presents itself as a potent diagnostic tool due to its low measurement times and sensitivity but is currently limited in scope due to the lack of multiplexed detection. The diagnostic utility of a single biomarker measurement is much less than a measurement of multiple markers.² A differential bead counting device (see, e.g., FIG. 4), relies on two key principles, coulter counting and specific capture. At the coulter counter (41 42), current passes through a narrow aperture 140 (typical commercially available aperture sizes range from 20 μm to 2000 μm), but is obstructed as particles flow past, displacing a volume of conductive electrolyte solution and correspondingly changing the impedance of the medium. For a particle less electrically conductive than the surrounding solution, the impedance increases. These spikes in impedance are counted as beads. In the specific capture chamber 63, beads are retained by the device in response to a sandwiched biomolecule assay performed on bead. Specific binding moieties (antibody, oligonucleotide sequence, aptamer, etc.) on the surface of the bead capture the target biomolecules from solution. A secondary probe with a biotin tag is introduced that binds to the captured biomolecules on the bead, forming a target molecule dependent link between the bead and the biotin capture functionality. The surfaces of the capture chamber are functionalized with streptavidin which binds biotin with high affinity³ enabling the robust capture of beads in response to the presence of the target biomolecule. By counting the beads as they enter and exit the capture chamber, the percent capture is obtained which can be used for quantification and detection.

To achieve multiplexed protein detection using differential counting technology it is important to design microparticles that can be captured uniformly using biotin-streptavidin interactions, in response to each unique immunoassay while at the same time producing distinct electrical impedance signatures needed for identification during counting. To produce significant impedance signatures the particle should occupy a substantial fraction of the volume of the detector aperture. As such, only a narrow range of sizes are accessible to any given coulter counter, because larger particles will obstruct flow and smaller particles may pass undetected. Although the capture chamber can be optimized for beads of any size, the simultaneous capture of multiple electrically distinct beads limits multiplex capture to a single geometry and flow speed. This further restricts the available range of sizes and densities that are suitable for multiplex particles because the beads must move slowly enough to interact with the streptavidin coated pillars without having a high rate of non-specific capture due to settling. Beads with highly distinct sizes or densities flow through the capture chamber very differently, resulting in either poor capture efficiency or high non-specific capture. For example, when using a lower flow rate that optimally captures (6-10 μm) commercially available solid magnetic beads, many of the larger (10-14 μm) commercially available magnetic beads will settle inside the device resulting in high non-specific capture. Similarly, when the flow speed is optimized for the larger solid beads, the smaller beads flow too quickly and are captured with low probability even when highly conjugated to biotin due to insufficient interaction with the capture surfaces. As such, there is a demand for multiple populations of electrically distinct microparticles, spanning a narrow range of sizes that are magnetic, surface functionalized, and can be captured selectively and simultaneously in response to unique biomolecular assays.

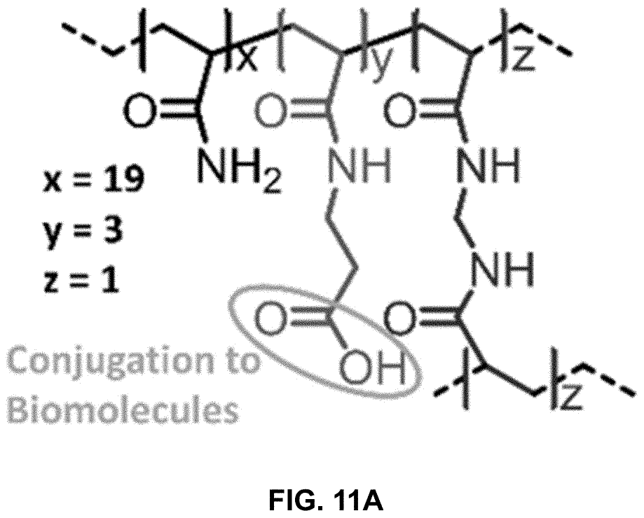

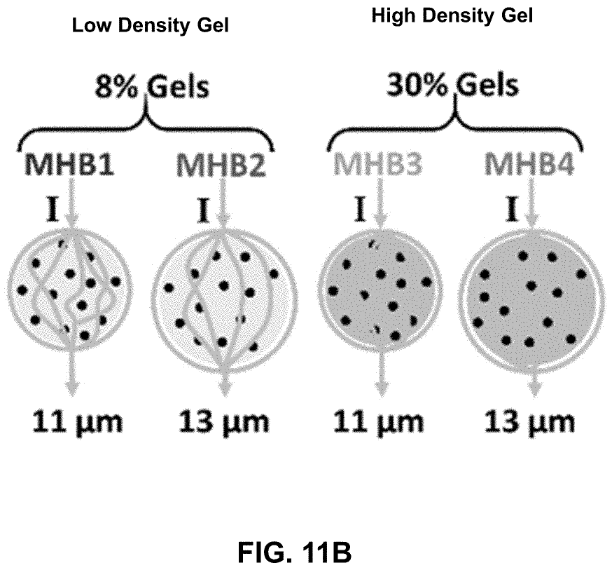

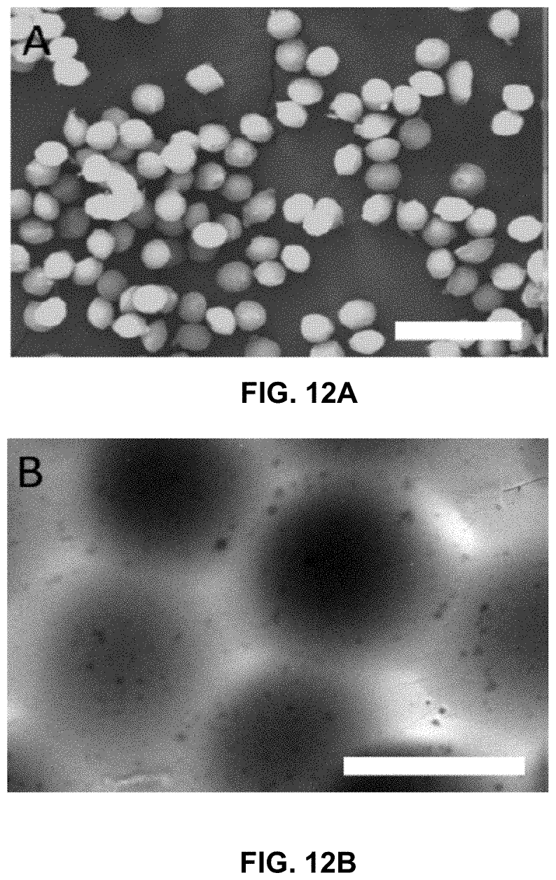

To accomplish this demanding synthesis, we utilize droplet microfluidic techniques to produce beads featuring this never before realized combination of properties. Droplet microfluidics is an effective method to create functional microparticles due to the robust generation of large numbers of highly monodisperse droplets which are used to template the polymerization of nearly identical hydrogels.⁴⁻⁶ A wide variety of chemical functionalities and material properties can be achieved using different monomers and polymerization schemes.⁷ Polyacrylamide is selected as the hydrogel scaffold for this application due to its fast and simple polymerization chemistry, biocompatibility, thermostability, and its relatively low viscosity. Conjugation of biomolecules to the hydrogel surface is provided by incorporating a modified acrylamide monomer with a free carboxylic acid moiety as shown in FIG. 11A. This monomer facilitates straightforward conjugation of antibodies or other biomolecules to the hydrogel surface using established amide coupling reactions.⁸ To render the hydrogel microparticles magnetic, magnetic nanoparticles are suspended in solution along with the monomers and crosslinkers. The composition of the resulting polyacrylamide gel is tuned such that the average pore size is significantly smaller than the diameter of the nanoparticles⁹, thereby physically trapping the particles in the hydrogel network during polymerization.

The impedance measurement recorded as a particle passes a coulter counter, results from the difference in electrical conductivity of the solution and the particle. The electrolyte buffer is highly conductive while the bead material acts as an insulator. When a bead occupies the region between the electrodes it displaces the conductive medium resulting in a change in impedance proportional to its volume. Compared to solid particles of the same size, hydrogel beads produce less impedance as current is partially able to flow through the swelled fraction of the hydrogel. As a result, the smallest detectable hydrogel bead for any specific coulter counter is larger relative to a solid bead. Despite this, hydrogels are advantageous for two reasons: 1) Microfluidic synthesis enables greater monodispersity and 2) Hydrogel beads have a density similar to water making them less susceptible to settling. To produce distinct impedance signatures, the size and the gel density of the hydrogel beads are varied as shown in FIG. 11B.

Using these principles, we synthesize monodisperse populations of hydrogel beads with tightly controlled sizes in the range of 10-14 μm, in this example four populations, with each population comprising magnetic particles. To create these hydrogel particles, acrylamide monomers, crosslinkers, iron oxide nanoparticles, and a radical initiator (ammonium persulfate) are added to the aqueous phase, which is encapsulated into drops (FIG. 2A). The drops are collected off-chip and heated to 90° C. overnight to ensure complete polymerization. The droplet emulsion remains monodisperse during polymerization (FIG. 2B). Afterwards, the oil is removed, and the hydrogel particles are washed and dispersed in buffer. The hydrogel particles absorb water and swell considerably; as such, the drop size must be tuned to produce the correct sized beads after swelling. The final populations of magnetic hydrogels are monodispersed with a coefficient of variation of the diameter of <5% (FIG. 2C). We confirm the incorporation of the magnetic nanoparticles by separating the gels from solution using a magnet. We observe suspension of hydrogels in PBS immediately after mixing by vortex, with one tube containing hydrogels without magnetic nanoparticles and another tube contains hydrogels with magnetic particles. After 5 minutes the magnetic hydrogels are collected on the magnet and can be separated from solution, with an observable difference in solution color. Precise control of drop size is achieved by varying flow rates of the two phases enabling the production of all four populations using a single device design. Minimizing the size difference is important to ensure similar flow rate and capture efficiency in the differential counting device. 11 μm and 13 μm polyacrylamide hydrogel particles are produced at ˜8% w/v. These two samples are denoted as MHB1 and MHB2, respectively. The optical images of MHB1 and MHB2 are shown in FIGS. 2D-2E. We experimentally determined that equally sized polyacrylamide hydrogels made at ˜30% w/v (an increase of 3.75 times) produce clearly distinguishable impedance signatures in our configuration. As such, populations of 11 μm and 13 μm hydrogel particles at this higher gel density are produced. These samples, respectively labeled MHB3 and MHB4 and shown in FIGS. 2F-2G. Although MHB1 and MHB3 are electrically distinguishable, MHB3 produces an impedance signature that overlaps with the larger but less dense MHB2. Together 3 electrically distinct populations of hydrogel beads are produced as shown in FIGS. 3A-3B. MHB2 is selected over MHB3 for further experiments due to its higher rate of production.

To demonstrate the successful carboxyl-functionalization of the hydrogel beads, each population is conjugated to amine-functionalized biotin and then mixed with fluorescently-labeled streptavidin to enable the visualization of conjugation. Using a flow cytometer, the fluorescent intensity of the beads is measured (FIG. 3C). These results demonstrate that the MHBs incorporate carboxylic acid moieties in the polymer network and that these functional groups can be used for further conjugation via amide coupling. Notably, the amount of carboxyl groups on each hydrogel bead can be easily tuned by adjusting the concentration of the carboxyl-modified monomers. As such we have synthesized each MHB to display substantially more carboxyl groups compared commercially available solid beads (FIG. 3C). Confocal imaging was performed to localize the fluorescent signal within each bead. FIG. 3D provides magnetization plots that confirm the produced hydrogels are magnetic and have successfully incorporated the magnetic nanoparticles inside. FIG. 3E shows the orthographic projection of the fluorescent signal of a single bead. The fluorescent dyes are located in a thin shell at the surface of the bead roughly 1-2 μm deep. Since capture depends on exposed biotin on the surface of the bead, these results importantly confirm that the majority of conjugation events occur at the surface and are able available to increase the probability of capture. FIG. 3F shows the three-dimensional reconstruction of a fluorescently labeled hydrogel bead from the z-stack confocal images. These images confirm the spherical shape of the beads.

We test the capture performance of these beads in the microfluidic differential counter (see, e.g., FIG. 4) by flowing biotin conjugated MHBs through the streptavidin coated capture chamber and counting the number of MHBs entering and exiting the device. This acts as a model for the multiplex immunoassays which results in the shared biotin tagging of each bead in proportion to the concentration of unique target proteins in the sample. FIG. 5A shows the dependence of capture percentage on the amount of biotin conjugated to the surface of each bead. This indicates that capture occurs sensitively in response to the biotin-streptavidin interaction.

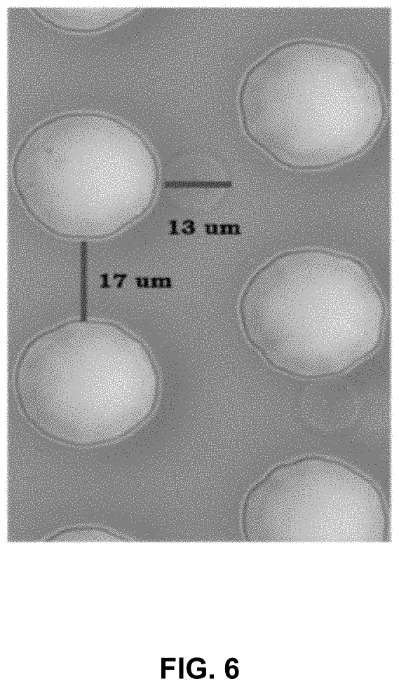

Importantly, capture is also selective as shown in FIG. 6. Only the MHBs that are conjugated to biotin are captured with high probability even as all 3 populations are flowed and counted simultaneously.

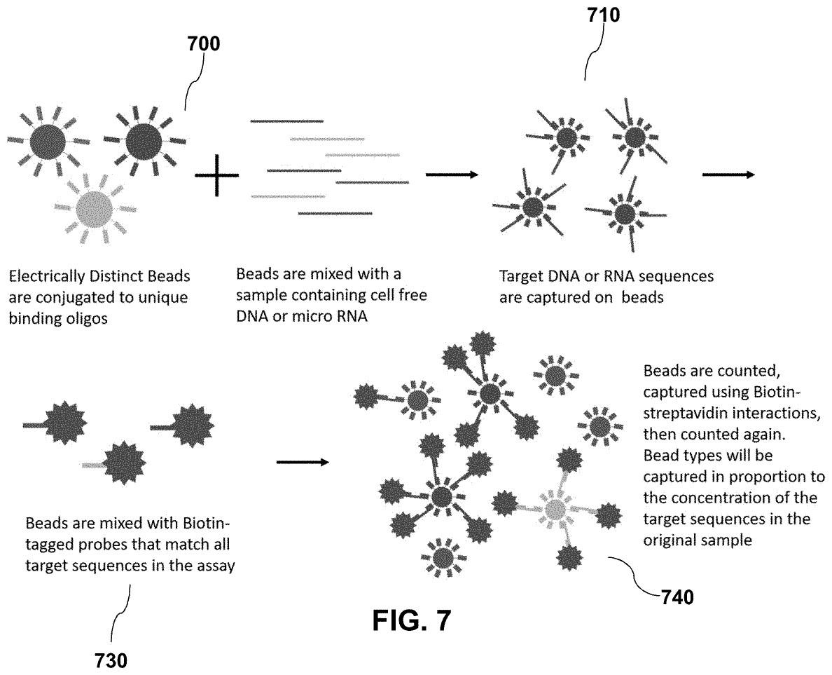

To demonstrate the multiplexed detection of biomolecules using the electrically distinct MHBs, short DNA fragments are selected. These short sequences are found in elevated levels in patients with cancer. The specific mutations are highly cancer specific and can act as markers for the disease and its progression.¹⁰ Three short (<70 nt) sequences are selected that are indicative of colorectal cancer. Each population of beads is conjugated to a ˜20 bp oligo that specifically targets one of the three selected fragments. The beads are then able to capture the DNA fragments from a sample solution. Finally, a set of 3 biotin tagged probes are introduced that bind to all the target fragments captured by the beads. In this way the beads are linked to biotin via the target fragments. Using this configuration we are able to detect the presence of each fragment from a sample solution through differential counting and capture of the electrically distinct beads. See, e.g., FIG. 7.

Using droplet microfluidics, we have designed, synthesized, and tested multiple monodisperse populations of magnetic, carboxyl-functionalized, electrically distinguishable, polyacrylamide hydrogel beads. Using a differential counting device, we confirmed the simultaneous and selective capture of each bead population and in proportion to the biotin conjugation of the hydrogel. These newly synthesized beads overcome the challenges associated with multiplexing the bead counting platform and demonstrate the potential for rapid quantification of multiple protein biomarkers from a patient sample.

### REFERENCES

Xi, H.-D., Zheng, H., Guo, W., Gañán-Calvo, A. M., Ai, Y., Tsao, C.-W., . . . Tan, S. H. (2017). Active droplet sorting in microfluidics: a review. Lab on a Chip, 17(5), 751-771. https://doi.org/10.1039/C6LC01435F

Valera, E., Berger, J., Hassan, U., Ghonge, T., Liu, J., Rappleye, M., . . . Bashir, R. (2018). A microfluidic biochip platform for electrical quantification of proteins. Lab on a Chip, 18(10), 1461-1470. https://doi.org/10.1039/c81c00033f

Liu, Y., Hou, J., Li, Q., Chen, K., Wang, S.-N., & Wang, J. (2016). Biomarkers for diagnosis of sepsis in patients with systemic inflammatory response syndrome: a systematic review and meta-analysis. SpringerPlus, 5, 2091. https://doi.org/10.1186/s40064-016-3591-5

Cohen, L., & Walt, D. R. (2018). Highly Sensitive and Multiplexed Protein Measurements. https://doi.org/10.1021/acs.chemrev.8b00257

Reslova, N., Michna, V., Kasny, M., Mikel, P., & Kralik, P. (2017). xMAP Technology: Applications in Detection of Pathogens. Frontiers in Microbiology, 8, 55. https://doi.org/10.3389/FMICB.2017.00055

Droplet microfluidics hydrogel synthesis papers

Chung, I. D., Britt, P., Xie, D., Harth, E., & Mays, J. (2005). Synthesis of amino acid-based polymers via atom transfer radical polymerization in aqueous media at ambient temperature. Chemical Communications, 0(8), 1046-1048. https://doi.org/10.1039/b416591hhttps://doi.org/10.1039/b416591h

1. Valera, E., Muriano, A., Pividori, I., Sánchez-Baeza, F., & Marco, M. P. (2013). Development of a Coulombimetric immunosensor based on specific antibodies labeled with CdS nanoparticles for sulfonamide antibiotic residues analysis and its application to honey samples. Biosensors and Bioelectronics, 43(1), 211-217. https://doi.org/10.1016/j.bios.2012.12.017

2. Dolin, H. H., Papadimos, T. J., Stepkowski, S., Chen, X., & Pan, Z. K. (2018). A Novel Combination of Biomarkers to Herald the Onset of Sepsis Prior to the Manifestation of Symptoms. Shock (Augusta, Ga.), 49(4), 364-370. https://doi.org/10.1097/SHK.0000000000001010

3. Chivers, C. E., Crozat, E., Chu, C., Moy, V. T., Sherratt, D. J., & Howarth, M. (2010). A streptavidin variant with slower biotin dissociation and increased mechanostability. Nature Methods, 7(5), 391-393. https://doi.org/10.1038/nmeth.1450

4. Kim, J. W., Utada, A. S., Fernández-Nieves, A., Hu, Z., & Weitz, D. A. (2007). Fabrication of monodisperse gel shells and functional microgels in microfluidic devices. Angewandte Chemie—International Edition, 46(11), 1819-1822. https://doi.org/10.1002/anie.200604206

5. Islam, M. M., Loewen, A., & Allen, P. B. (2018). Simple, low-cost fabrication of acrylic based droplet microfluidics and its use to generate DNA-coated particles. Scientific Reports, 8(1), 8763. https://doi.org/10.1038/s41598-018-27037-5

6. Teh, S. Y., Lin, R., Hung, L. H., & Lee, A. P. (2008, Jan. 29). Droplet microfluidics. Lab on a Chip. The Royal Society of Chemistry. https://doi.org/10.1039/b715524g

7. Liu, E. Y., Jung, S., Weitz, D. A., Yi, H., & Choi, C.-H. (2018). High-throughput double emulsion-based microfluidic production of hydrogel microspheres with tunable chemical functionalities toward biomolecular conjugation. Lab on a Chip. https://doi.org/10.1039/C7LC01088E

8. Sehgal, D., & Vijay, I. K. (1994). A method for the high efficiency of water-soluble carbodiimide-mediated am idation. Analytical Biochemistry, 218(1), 87-91. https://doi.org/10.1006/abio.1994.1144

9. Holmes, D. L., & Stellwagen, N. C. (1991). Estimation of polyacrylamide gel pore size from Ferguson plots of linear DNA fragments. Electrophoresis, 12(9), 612-619. https://doi.org/10.1002/pssb.201300062

10. Diehl, F., Schmidt, K., Choti, M. A., Romans, K., Goodman, S., Li, M., Diaz Jr, L. A. (2008). Circulating mutant DNA to assess tumor dynamics. Nature Medicine, 14(9), 985-990. https://doi.org/10.1038/nm.1789.

### EXAMPLE 7

### Rapid, Multiplexed Detection of Biomolecules using Electrically Distinct Hydrogel Beads

Rapid, low-cost, and multiplexed biomolecule detection is an important goal in the development of effective molecular diagnostics. We have demonstrated a microfluidic biochip device that can electrically quantitate a protein target with high sensitivity. This platform detects and quantifies a target analyte by counting and capturing micron-sized beads in response to an immunoassay on the bead surface. Existing microparticles limit the technique to the detection of a single protein target and lack the magnetic properties required for separation of the microparticles for direct measurements from whole blood. Here, we report new precisely engineered microparticles that achieve electrical multiplexing and adapt this platform for low-cost and label-free multiplexed electrical detection of biomolecules. Droplet microfluidic synthesis yielded highly-monodisperse populations of magnetic hydrogel beads (MHBs) with the necessary properties for multiplexing the electrical Coulter counting on a chip. Each bead population is designed to contain a different amount of the hydrogel material, resulting in a unique electrical impedance signature during Coulter counting, thereby enabling unique identification of each bead. These monodisperse bead populations span a narrow range of sizes ensuring that all can be captured sensitively and selectively under simultaneously flow. Incorporating these newly synthesized beads, we demonstrate versatile and multiplexed biomolecule detection of proteins or DNA targets. This development of multiplexed beads for the electrical detection of biomolecules, is important for multiplexing of the Coulter counting approach and the development of a low cost point-of-care diagnostic sensor.

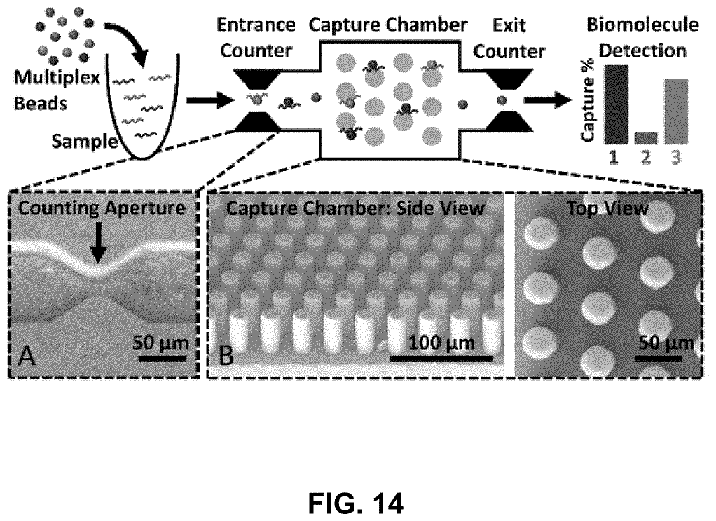

Accurate and timely monitoring of biomarker levels can dramatically improve the treatment of disease by improving diagnosis.¹⁻⁷ It is well understood that the combination of multiple biomarker measurements results in increased predictive and diagnostic power when compared to a single measurement.⁸⁻¹⁴ Accordingly, there is considerable interest in the development of improved methods for multiplexed biomarker screening. Existing methods for multiplexed biomarker measurement typically require chemical labeling schemes that encode the presence and quantity of each biomolecule target using color and intensity of light,¹⁵⁻¹⁹ or using electrochemical^{20,21} or mass spectrometry -based detection.²² However, those techniques typically require complex, expensive instrumentation and technical expertise to achieve multiplexing, and are not suitable for widespread clinical adoption. Label free electrical detection of biomolecules presents a promising, low-cost alternative. Previous work has demonstrated a strategy of protein quantification using a coulter counter that relies on biomolecule mediated bead aggregation to increase particle impedance in response to the quantity of the target.²³ However, that technique requires the full range of impedance signals to quantify a single biomolecule target, making it inherently singleplex. Our recent work has demonstrated a simplified alternative method that requires only a limited impedance range to electrically quantify either cells or a single protein target.²⁴⁻²⁶ This differential counting technology requires three components connected in series: an entrance Coulter counter, a capture chamber, and an exit Coulter counter (FIG. 14). When detecting proteins, the device enumerates the total number of micron-sized particles that enter by electrical counting. Subsequently, these beads are captured on an array of pillars in proportion to the target analyte, using a sandwich-immunoassay, before the remaining uncaptured beads are counted as they pass the exit counter. The sandwiched immunoassay that takes place on the surface of each bead, tags the bead with biotin capture groups in proportion to the target analyte. This biotin increases the likelihood of bead capture which is measured by the difference in bead counts at the entrance and exit of the device. Using this platform, we demonstrated physiologically significant and sensitive detection of IL-6, a well-studied sepsis biomarker, (LOD: 127 pg/mL).²⁴ Despite its potential for further multiplexing, a lack of suitable microparticles limited this technique to the detection of a single target. Moreover, the beads used in this iteration of the device were not magnetic. Magnetic beads enable facile separation from cells which would be useful for a combined analysis of cells and proteins from whole blood.

Multiplexed bead counting can be achieved straightforwardly by varying the size of the microparticles to result in distinct electrical signals.²⁷ However, differently sized particles experience a different flow profile inside the device impacting the likelihood of capturing each bead. Multiplexed biomolecule detection requires a uniform capture response across multiple different beads on a single device. Accordingly, the size differences between each bead population must be minimized so that each bead type interacts with the capture pillars similarly and a proportional and sensitive capture response is obtained across all populations. Although microparticles of various sizes and material properties are available commercially, they are not currently designed with their impedance signal in mind. Consequently, there is lack of microparticles with the key set of properties required for multiplexing. Solid magnetic beads tend to have broad size distributions which necessitates even larger differences in size to obtain unique, non-overlapping populations. As a result, those particles fail to respond uniformly during multiplexed bead capture. To address the challenges and achieve electrical multiplexing, precisely engineered microparticles are needed. We synthesized new tightly spaced, monodisperse magnetic hydrogel bead populations that produce distinct impedance signatures to enable multiplexed electrical biomarker monitoring. A schematic overview of one embodiment of a multiplex device for use with a multiplex particle system (e.g., “multiplex beads”) is outlined in FIG. 14.

In summary, suitable microparticles for electrical multiplexing are selected to satisfy the following: (i) Each bead population must yield distinct electrical impedance signatures so that all beads can be uniquely identified during Coulter counting. Since the impedance of a particle measured by a Coulter counter is proportional to the volume of the conductive medium that it occupies, each distinct population must displace a unique volume of electrolyte.²⁸ (ii) All populations should fit within a narrow range of sizes so that the capture efficiency of each bead is uniform and nonspecific capture is low across all beads. (iii) Each bead population must also have useful surface chemistry. Surface functional groups are needed to conjugate targeting biomolecules such as antibodies, aptamers, DNA sequences, etc. These targeting biomolecules are used to convert increased biomarker levels into an increased probability of bead capture through a sandwich assay. (iv) Magnetic properties are also necessary for on-chip manipulations of the beads. In addition, these magnetic properties allow for an additional level of multiplexing through an orthogonal measurement of non-magnetic microparticles. As an example, cells, which would otherwise produce overlapping impedances with similarly sized beads, could be counted and resolved by the device by using a magnet to isolate the two measurements. In this way, magnetic properties would enable cell and protein measurements on a single platform. Our previous publications,^{25,26} details a method of leukocyte counting and cell surface antigen-mediated cell capture that could be readily combined with a measurement of magnetic bead capture for multiplexed cell and biomolecule detection.

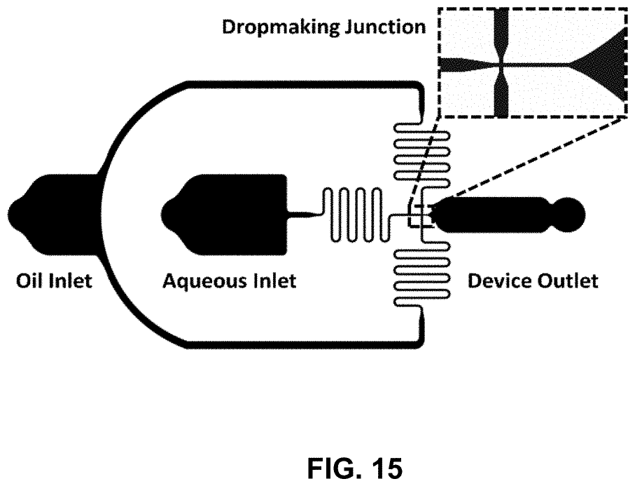

Fabrication of Microfluidic Devices. The electrical bead counting devices and capture chambers were fabricated according to previously detailed procedures.²⁴ SEM images of the Coulter counting aperture and capture chamber were collected using a Hitachi S-2250N. Prior to imaging a layer of gold was sputtered to coat the capture chamber. The Microfluidic drop-making devices were prepared using standard photolithography techniques. Device masters were prepared using SU-8 2010 (MicroChem) negative photoresist to coat a 3-inch silicon wafer to a height of 10 microns. The device structures (FIG. 15) are patterned onto the photoresist using standard photolithography techniques. PDMS prepolymer and curing agent (Sylgard) are mixed in a 10:1 ratio and poured to coat the device master. The PDMS is degassed then cured at 90° C. for at least 1 hour. Cured PDMS slabs are cut from the master, and device ports are cast using a 1 mm biopsy punch (TedPella Inc.). Prior to bonding, the surfaces of the PDMS and glass slide (Corning) are activated for 10-15 seconds using a 30 W plasma cleaner (Harrick Plasma). The PDMS is pressed onto the glass slide forming an irreversible bond. The channels of the device are flushed with Aquapel (PPG Industries) and then purged with Nitrogen rendering the surface hydrophobic.

Hydrogel Synthesis. Polyacrylamide-based hydrogels are prepared using a flow-focusing droplet generation device. The carboxyl-modified monomer, Acryloyl β-alanine (ABA), is synthesized from acryloyl chloride and β-alanine (Sigma Aldrich) and confirmed by ¹H NMR (collected on a Varian Inova 400). Acrylamide and bis-acrylamide are purchased from BioRad. The dispersed phase, a gel forming mixture consisting of 19:1 acrylamide monomers and crosslinkers, 1% ammonium persulfate and suspended magnetic nanoparticles (200 nm silica-coated, Creative Diagnostics) at a concentration of 5×10⁹ particles/m L, is prepared immediately prior to device operation. The continuous phase contains 2% 008-FluoroSurfactant (Ran Biotechnologies) in HFE-7500 fluorinated oil (3M) and is passed through a 0.2 μm PTFE syringe filter to exclude debris. Suitable flowrates that yielded 11 and 13 μm beads are determined experimentally. Smaller drops (˜8 μm) re produced at 15:125 μL h⁻¹ aq:oil which would swell to 11 μm in 1× PBS. Larger drops (˜10 μm) are produced at 25:75 μL/h aq:oil which would swell to 13 μm in 1× PBS. The resulting droplets are collected in a 1.5 mL tube under mineral oil, which prevents evaporation. The emulsion is then heated to 90° C. to induce polymerization. The hydrogels are fully solidified after 1 hour. The magnetic hydrogels are released from the droplets by the addition of an excess of 20% 1H,1H,2H,2H-Perfluoro-1-octanol (Fisher Scientific) in HFE. The tube is vortexed and centrifuged. Then the fluorinated oil is removed. The hydrogels are washed once with hexanes to dissolve any unreacted monomers then washed 3 times with 0.2% Tween-20 in 1× PBS.

Characterization of Bead Properties. ζ-potentials are measured on a Malvern Zetasizer Nano ZS (Malvern Instruments). A dilute bead suspension (1×10⁵ beads mL⁻¹) in 0.01 M PB buffer (pH 7.4) is loaded into a disposable folded capillary cell and measured. Values are reported as the average of triplicate runs consisting of 100 scans each. To obtain transmission electron microscope (TEM) images, the hydrogels are washed 3 times with water to remove excess salts. 5 μL of a solution containing dispersed hydrogels are spotted on a TEM grid and freeze-dried. Samples are imaged at 20 kV using a JEOL 2010 Lab6 TEM. The bulk magnetic properties of each population of magnetic hydrogels is assessed using a Vibrating Sample Magnetometer (VSM). The magnetization of the hydrogels in response to an applied external magnetic field is detected as a current in an inductively coupled superconducting detection coil which is converted to voltage using a superconducting quantum interference device (SQUID). The SQUID output voltage is monitored as the applied field is varied from 10 kOe to −10 kOe measuring the magnetic response of the sample. The SQUID measurements are obtained at 5K and 300K for each sample.

Magnetic separation from cells is performed as follows; U-2 OS cells are trypsinized then resuspended in a 2 μg mL⁻¹ solution of Hoechst dye at 37° C. for 15 mins. These cells are resuspended in PBS and complete staining is confirmed by imaging. Stained cells and beads were mixed in an approximate 1:1 ratio and imaged again. The mixed suspension of beads and cells is placed on a magnet and the solution. The remaining beads are resuspended in PBS and imaged again. Electrical measurements re obtained on a custom chip and analyzed as described previously.²⁴ Fluorescent labeling of biotinylated beads is achieved using a 6.7 μg mL⁻¹ solution of streptavidin, R-phycoerythrin conjugate (Fisher Scientific). The beads are incubated for approximately 15 minutes then washed to remove unbound dyes. Confocal images are obtained using a Zeiss 710 confocal scanner, an Axio Observer Z1 microscope, and a Spectra-Physics Ti:Sapphire laser. Other fluorescence measurements are obtained using a Guava easyCyte plus flow cytometer (Millipore).

Hydrogel Bead Functionalization. Biomolecules are conjugated to the bead populations using carbodiimide coupling chemistry. The beads are washed with MES buffer: 0.1 M 2-(N-morpholino)ethansulfonic acid (pH 6). Then resuspended in 1 mM 1-Ethyl-3-(3-dimethylaminopropyl)carbodiim ide (EDC) with 0.5 mM N-hydroxysulfosuccinimide (NHS) in MES and mixed for 15 min to activate the carboxyl groups on the bead. The beads are centrifuged or separated using a magnet and the solution is removed. The amine-containing molecule is then added, and the beads are mixed for 2 hours enabling covalent coupling through amide bond formation. For biotin functionalization, 4 or 20 μg mL⁻¹ NH₂-(PEG)₂-Biotin (Fisher Scientific) in PBS (pH 7.4) was used. For antibody functionalization, 0.5 mg mL⁻¹ Ab1IL6 (Fisher) or Ab1PCT (Abbexa) are used. For DNA functionalization, 90 μM DNA capture oligos with 3′ amine modification (IDT) are used. Following conjugation, unreacted biomolecules are removed during three washes with PBS. The functionalized hydrogels are then resuspended in a 1% BSA in PBS solution to block nonspecific interactions between the bead surface and the capture pillars.

Sandwiched Assays for Multiplexed Biomolecule Measurements. The multiplexed biomolecule assays are adapted from our previously described protocol.²⁴ For protein detection, MHB1, MHB2-Ab1IL6, and MHB4-Ab1PCT are mixed equally and incubated with a 500 ng mL⁻¹ IL-6 (Shenandoah). The solution is removed, and the beads resuspended in 5 μg mL⁻¹ Ab2L6-biotin and Ab2PCT-biotin probes to label proteins on the beads with biotin capture tags. For DNA detection, MHB1, MHB2-TP53CaptureOligo, MHB4-PIK3CACaptureOligo, are mixed equally and incubated in 15 μM TP53 gene fragments. Next, the solution is removed, and the beads resuspended in 50 μM biotin labeled probes specific to each fragment in the assay. The solution is then heated to 65° C. then slowly cooled to 37° C. for sequence annealing. The sequences used in this protocol (Integrated DNA Technologies) are included as supplementary data. The prepared beads from each sandwiched assay are washed 3 times in PBS to remove any unbound probes prior to on-chip counting and capture.

Droplet Microfluidic Synthesis of Magnetic Hydrogel Beads. Using droplet microfluidic techniques, we synthesize magnetic hydrogel beads (MHBs) with the properties required for electrical multiplexing of the differential bead counting device. To ensure that each bead population is highly monodisperse, we leveraged established drop microfluidic synthesis methods.²⁹⁻³³ Microfluidics generates monodisperse droplets that are used to template polymerization, creating nearly identical functional microparticles. The droplet generation rate is on the order of 10 kHz resulting the high throughput generation of tens of millions of beads per hour. Precise control of the particle size is achieved by varying the flow rate of the two immiscible phases during droplet formation. The chemical and physical properties of the resulting beads are controlled through the contents of the solution that is encapsulated into droplets. We use a polyacrylamide-based hydrogel scaffold to produce the multiplex beads due to its fast and simple aqueous-phase polymerization chemistry, low viscosity, water solubility, biocompatibility, and thermostability. Additionally, polyacrylamide hydrogels absorb large volumes of water to more closely match the density of the solution in which they are suspended. This density matching reduces settling during measurement, which would otherwise be counted as false positive bead capture. Conjugation of biomolecules to the hydrogel is facilitated by the incorporation of a carboxylic acid moiety containing acrylamide monomer as shown in FIG. 11A. The carboxyl groups incorporated in the hydrogel can be conjugated to various capture groups using established amide coupling chemistry.^{34,35} We also include 200 nm magnetic nanoparticles in solution along with the monomers and crosslinkers, to impart magnetic properties to the beads. The crosslinking ratio and total monomer content were chosen to form gels with an average pore size less than 40 nm,³⁶ physically trapping the nanoparticles during polymerization.

As explained previously, the impedance signal produced by a microparticle corresponds to the volume of the conductive buffer it displaces. To synthesize beads with distinct impedance signatures, we vary the size and density of the hydrogel microparticles to produce beads with different solid volumes as shown in FIG. 11 B. Since these beads are hydrogels, electrical current (yellow lines) is partially able to flow through the buffer that fills the porous gel mesh. As such, beads with an increased gel density displace more conductive buffer without increasing the diameter of the microparticle. We produced 11 μm and 13 μm polyacrylamide hydrogel particles at ˜8% w/v of acrylamide, denoted as MHB1 and MHB2 respectively. Additional 11 μm and 13 pm polyacrylamide hydrogel populations are produced at ˜30% w/v which are respectively labeled MHB3 and MHB4. This size range is selected so that the beads are compatible with mammalian cell sizes for simultaneous measurements of cells and proteins in the future.

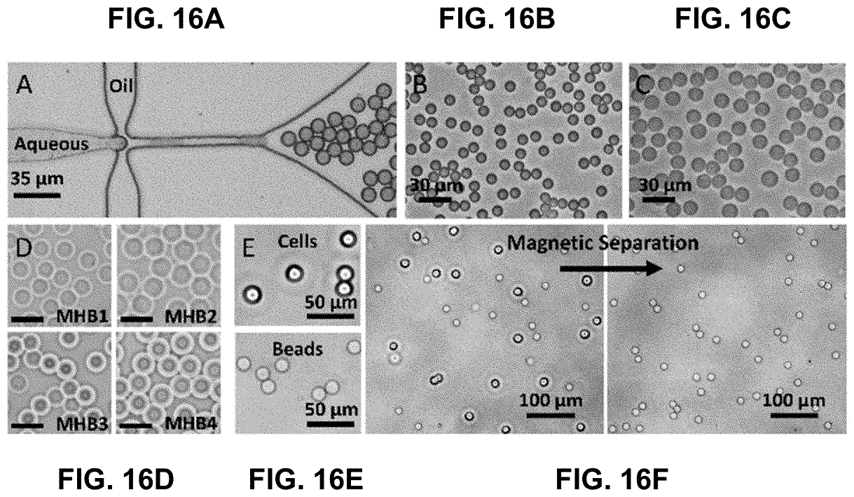

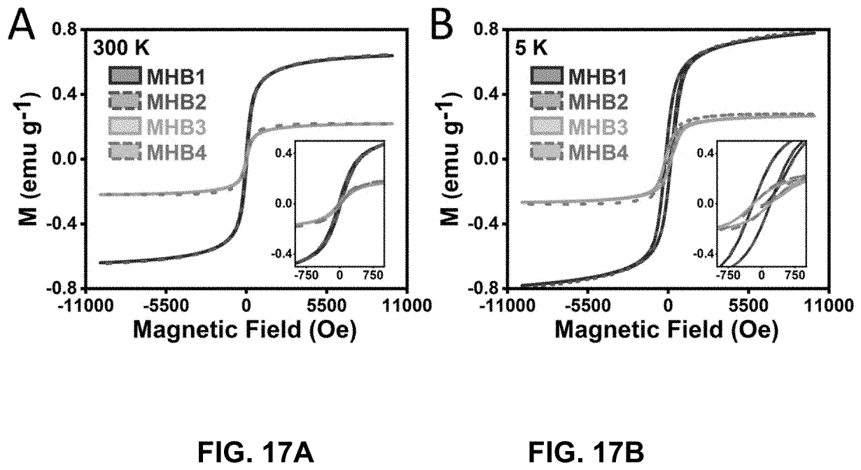

As shown in FIGS. 16A-16F, monodisperse populations of MHBs are synthesized using drop microfluidic templated polymerization. The aqueous phase, composed of acrylamide monomers, crosslinkers, iron oxide nanoparticles, and ammonium persulfate, is pinched-off into droplets by fluorinated oil using a flow-focusing drop-maker (FIG. 16A). The emulsion is collected off-chip and heated to facilitate polymerization. The surfactants in the fluorinated oil stabilize the droplet emulsion during heat induced polymerization preventing merging. (FIG. 16B). The emulsion is then broken, and the hydrogel particles are dispersed in buffer where they absorb water and swell considerably (FIG. 16C). The dispersed hydrogels remain monodisperse.

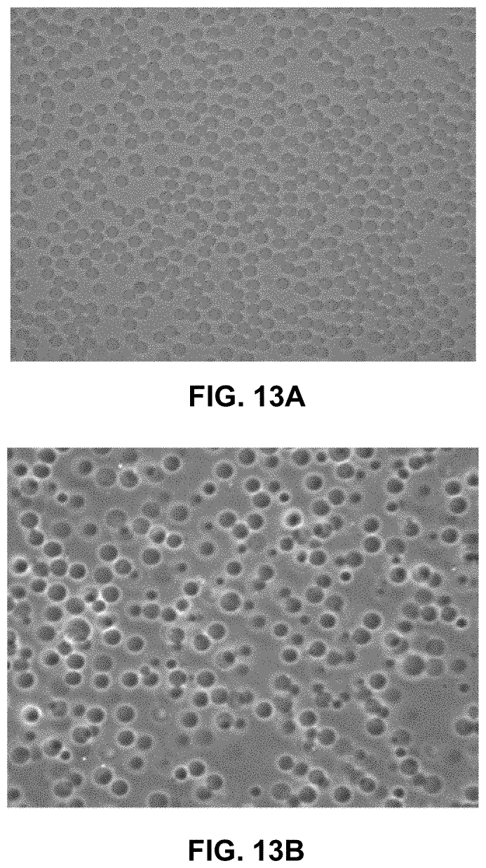

Physical Characterization of Bead Properties. We characterized the size, monodispersity, and morphology of the synthesized bead populations by microscope imaging and analysis. Optical images of the four synthesized MHB populations are shown in FIG. 13D (labeled MHB1-MHB4). Phase contrast was used to increase the visibility of the gels. The hydrogels are spherical in shape. MHB3 and MHB4 show heightened contrast compared to the lower density gels (MHB1 and MHB2) due to their increased gel content. The bead diameters of at least 100 hydrogels from each population were measured. MHB1 was synthesized with an average bead diameter of 10.9 μm and a standard deviation of 0.29 μm. MHB2 was 13.3±0.39 μm in diameter. Likewise, MHB3 and MHB4 had an average bead diameter of 11.1±0.33 μm and 13.1±0.38 μm respectively. These measurements confirm that each of the synthesized MHB populations are highly monodisperse with a coefficient of variation in the diameter of <3%. In this manner, the term monodisperse may refer to the relatively narrow distribution of the bead diameter over a specific population, including a coefficient of variation in the diameter of any given bead population that is less than 5%.

The swelling of the hydrogel beads was also assessed by comparing the droplet size and the corresponding bead size when dispersed in PBS. The volume of the swelled beads was found to be ˜2.2 times the droplet volume. This swelling factor was observed across bead types and is lower than the typical value for unmodified polyacrylamide microspheres.³⁷ The observed degree of swelling is likely reduced due to the salt concentration of the buffer solution (137 mM NaCl) and the ionic character of the hydrogel. Ionic polyacrylamide hydrogels are known to show suppressed swelling behavior at mM concentrations of NaCl.³⁸ The incorporation of ABA monomers is expected to yield a polyanionic gel in PBS (pH 7.4). The anionic character of the gels at pH 7.4 was confirmed by zeta potential measurements (−21.7±0.25 mV). Compact hydrogels are advantageous for Coulter detection due to their increased impedance relative to their size.

The magnetic properties of the synthesized beads were also assessed. TEM images of MHB1 following multiple washes show magnetic nanoparticles distributed throughout the gels confirming that the nanoparticles are trapped within the hydrogel during polymerization. The incorporation of these particles imparts magnetic properties to the beads so that the gels can be pulled from solution using an external magnet. To demonstrate a magnetic separation, we mixed the MHBs with fluorescently labeled cells (FIG. 16E). The mixed suspension of beads and cells was placed on a magnet and the solution was removed. The hydrogels were resuspended and imaged again to verify the magnetic separation. A SQUID magnetometer was used to measure the bulk magnetization and hysteresis behavior of the dried hydrogel beads (Figure S5) confirming that the hydrogel microparticles are paramagnetic. The saturation magnetization (Mₛ) of each sample was also obtained from the hysteresis plots. As an example, the Mₛ of MHB1 was 0.8 emu g⁻¹ which was used to estimate that each microsphere contained on average 20 magnetic nanoparticles. These experiments demonstrate the potential of these magnetic hydrogel beads for magnetic separation from cells (FIG. 16F), which is important for the direct analysis of protein biomarkers in blood.

Electrical Characterization of MHBs. The electrical impedance signatures of the bead populations were assessed by Coulter counting. The counter contains three microfabricated co-planar metallic electrodes aligned with the narrow apertures of a microfluidic channel. This impedance sensor design has been demonstrated to effectively measure the impedance of microparticles even at high flow speeds.³⁹ When a bead passes through the aperture, it displaces a large fraction of conductive buffer, producing a spike in impedance (a drop in the conductivity of the medium). FIGS. 3G-3I shows the differential impedance signal as the hydrogel beads flow past the electrodes of the Coulter counter. The biphasic impedance pulse enables particles with weaker signals to be more easily resolved from the noise.

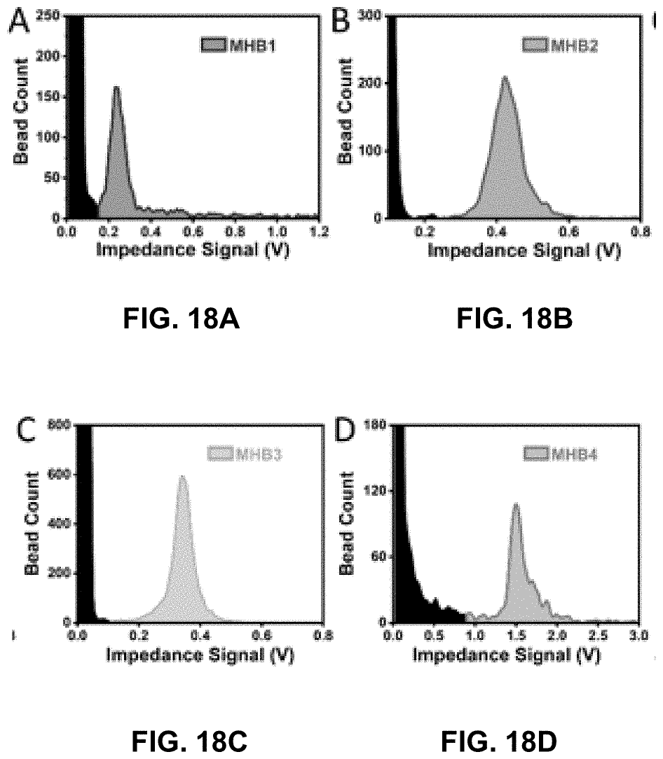

The electrical properties of the synthesized hydrogels were assessed using our microfluidic Coulter counter. MHB1 (FIG. 3G and FIG. 18A) was designed through its size (11 μm) and gel content to produce a minimal impedance signal that can still be readily resolved from the noise. MHB2 (FIG. 3H and FIG. 18B) was designed to be compositionally identical to MHB1 by just large enough (13 μm) to produce an impedance signal that does not overlap. Due to the monodispersity of the particles a minimal difference in size is sufficient to yield distinct impedance signatures. MHB3 FIG. 18C) likewise was designed to be the same size as MHB1 but to contain sufficient gel content to produce a non-overlapping impedance signature. MHB2 and MHB3 are not electrically distinguishable from each other but demonstrate orthogonal methods used to control the impedance signals produced by hydrogel particles during Coulter counting. MHB4 (FIG. 3I and FIG. 18D) was designed to produce the largest impedance signature by increasing both bead size and gel content. FIG. 3B shows the histogram of electrical impedance signals obtained from a mixed sample of MHB1, MHB2, and MHB4. The three distinct bead populations are clearly visible. FIG. 3A shows an example of the voltage reading of the impedance sensor as 3 beads pass the counter in sequence. During analysis, each recorded impedance spike is assigned to its corresponding bead population. These results demonstrate the successful synthesis of narrowly spaced particles for electrical multiplexing by Coulter counting.

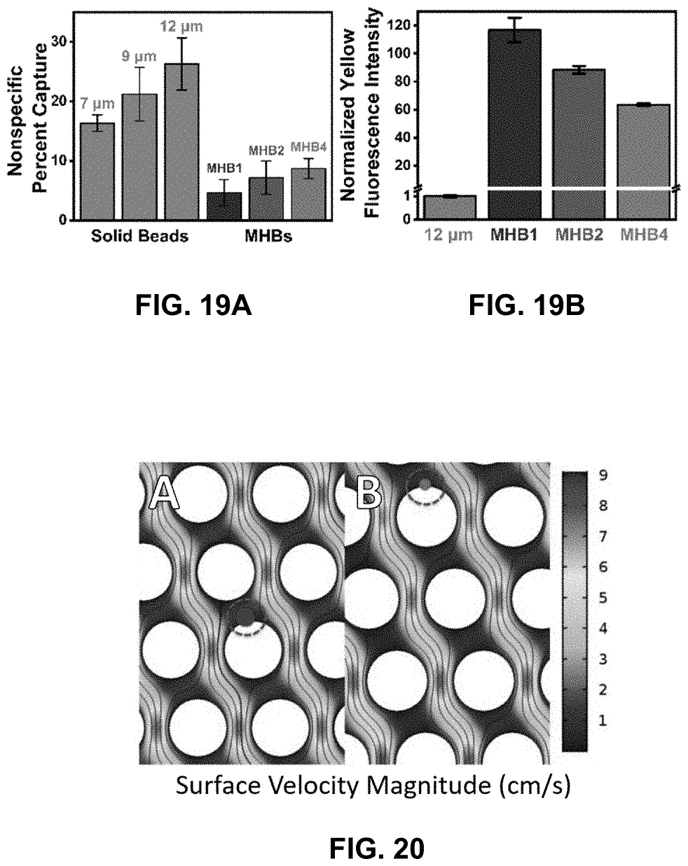

Comparison of MHBs and commercial magnetic beads for electrical multiplexing. Existing commercially available magnetic particles can provide distinct electrical signatures, but they cannot be used for multiplexed detection due to their behavior during capture. A uniform capture response across the set of electrically distinct particles is crucial to this method of multiplexed detection. Both a low degree of nonspecific capture and a high capture efficiency upon binding to the target are needed for sensitive multiplexing. FIG. 19A, shows a comparison of the nonspecific capture of various beads under a single flow condition (20 μL min⁻¹). Notably, the solid magnetic particles show high nonspecific capture across all populations, likely due to their increased density. To reduce this rate of non-specific capture a higher flow rate could be used (FIG. 20). However, increased flow rates also negatively impact the efficiency of specific capture. As an example from our previous work, the percent capture of CD8+ T cells (˜8 μm diameter) dropped from 90% to 40%, when the flow rate increased from 20 to 25 uL min⁻¹.²⁵ A similar inverse relationship between the rate of capture and the flow velocity has been observed independently in different device geometries.^{40,41} Additionally, higher flow rates negatively impact Coulter detection. As the flow rate increases the sampling depth of the impedance measurement is reduced, the signal to noise ratio decreases, and the baseline noise rises.²⁶ To balance nonspecific capture, capture efficiency, and electrical detection we use a flow rate of 20 μL min⁻¹ which has been shown as effective for sensitive capture of cells and solid (˜7 μm) latex beads.²⁴⁻²⁶ Accordingly, it is critical for a set of multiplexed beads to display a suitable capture response at this flow rate. The new MHB designs address the limitations of existing solid magnetic beads by exhibiting a low nonspecific capture across all populations, likely due to the closely matched density of hydrogels to the suspending buffer.

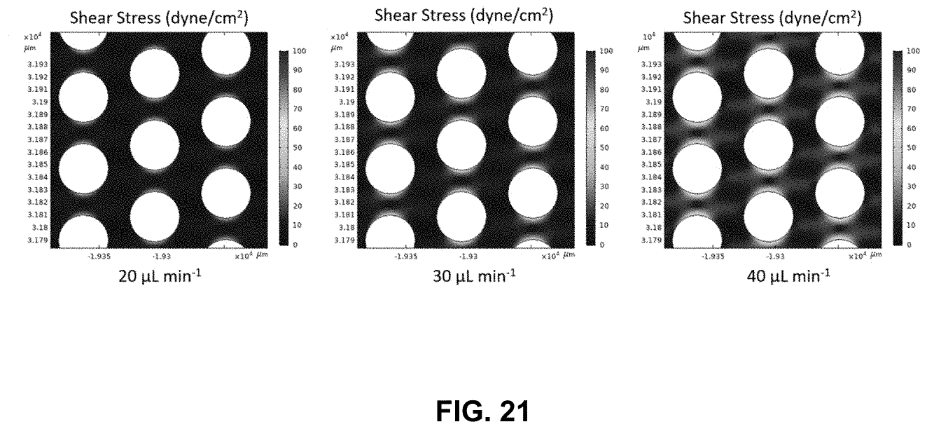

Another critical limitation of existing commercial particles is the variability in their capture response. In order to achieve 3 electrically distinct populations on our Coulter counter, we selected magnetic beads with average diameters of 7 μm, 9 μm, and 12 μm. Unfortunately, these solid magnetic beads are somewhat heterogeneous in size. As a result, a much larger range of diameters (6-14 μm) was needed to achieve the same level of electrical multiplexing as the set of MHBs (11-13 μm). Accordingly, the set of solid magnetic beads displayed considerable variability in the capture response at a shared flow rate. This result can be seen in FIG. 19A. The rate of nonspecific capture for each bead population was tested under the same flow conditions. The non-specific capture varies significantly across the electrically distinct solid bead populations while the MHBs show a consistent response. Particles of different sizes experience different flow profiles inside the capture chamber which impacts their capture response (FIG. 21). Accordingly, these solid magnetic beads respond differently to the same flow conditions, making them unsuitable for multiplexing.

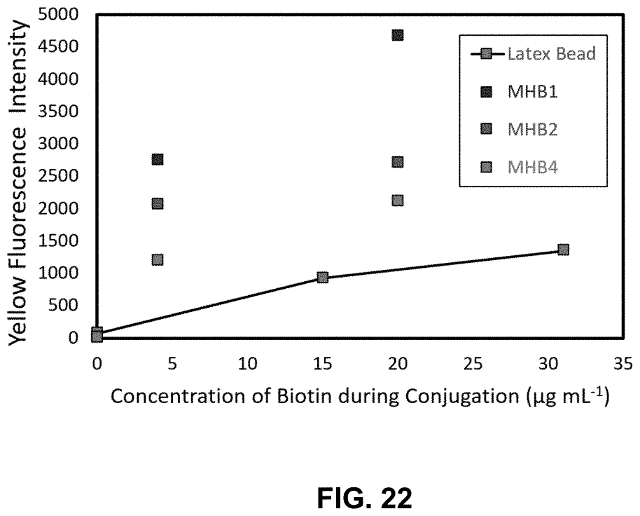

Lastly, surface functionalization of commercially available magnetic particles has limitations. The polyacrylamide synthesis method used to make the MHBs is highly modular. We can readily increase, decrease, or change the functional groups on the beads by adjusting the concentration or type of monomers during polymerization (generally referred herein as “tuning”). In contrast, the surface chemistry of commercial magnetic beads cannot be easily adjusted. FIG. 19B shows a comparison of the functional group density of the different beads. When conjugated to biotin under the same conditions, the hydrogel beads displayed substantially higher levels of surface functionalization. Because the hydrogels were designed to have a higher functional group density than the commercial beads, they allow for a greater degree of conjugation to biomolecules (FIG. 19B and FIG. 22). This increased functional group density enables a more sensitive response during biomolecular binding assays. From these results it is clear that these newly synthesized hydrogels address the current limitations in electrical detection and multiplexing.

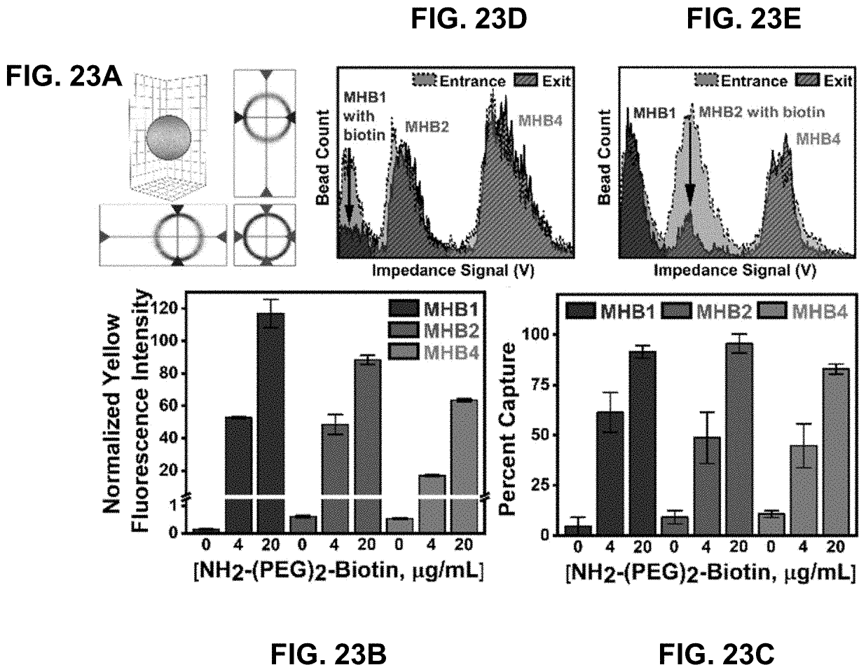

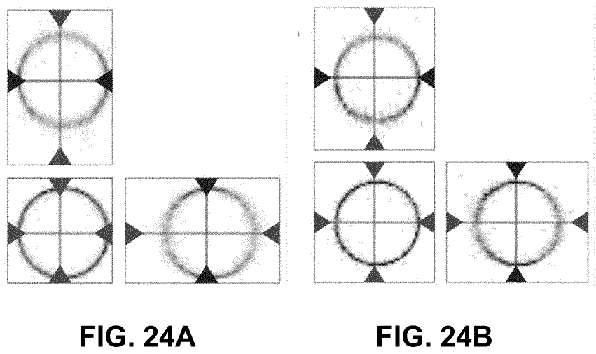

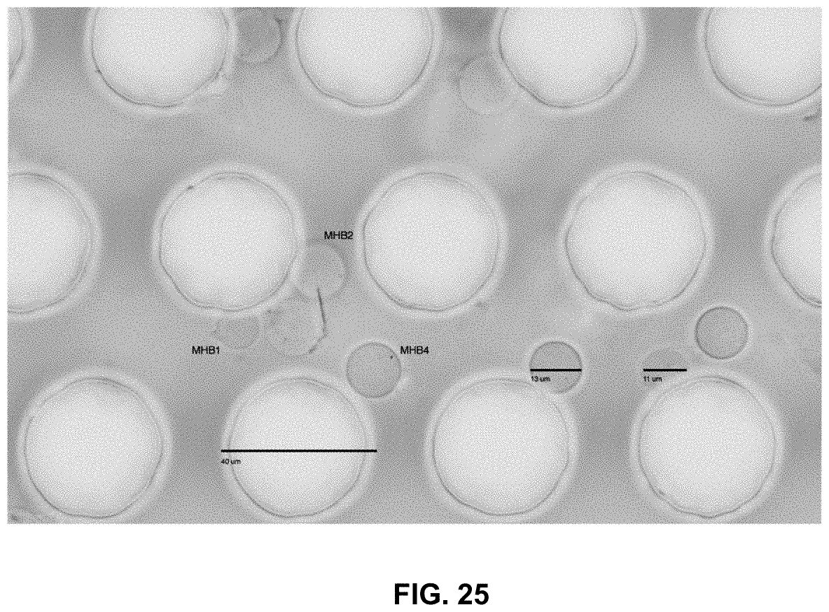

Sensitivity of Bead Capture to Surface Biotin. During the proposed multiplexed biomolecular sandwich assay, each bead type is linked to biotin-probes in proportion to the concentration of its target analyte. Since the capture chamber is functionalized with streptavidin, each of the individual assays can be detected using a shared capture mechanism. This same configuration can also use secondary antibodies to mediate capture; however, due to the higher affinity and specificity of the biotin-streptavidin interaction,^{42,43} it is selected for robust and sensitive bead capture. It is critical that each bead type displays a sensitive capture response to the biotin on its surface. In order to assess the relationship between the surface biotin and the bead capture, we conjugated each bead type to biotin (20 μg mL⁻¹) and labeled them with an excess of fluorescent streptavidin. The fluorescence of the functionalized beads was then measured by confocal imaging. The confocal images shown in FIG. 23A and FIG. 24 indicate that the fluorescent signal is localized in a thin fluorescent shell at the surface of each bead. The average thickness of the fluorescent region varies slightly between bead types: 1.8 μm for MHB1, 1.5 μm for MHB2, and 0.9 μm for MHB4. Importantly, any biotin that is conjugated inside the hydrogel cannot contribute to the capture, which occurs at the surface. but does not impact the capture response which depends only on the surface biotin. Accordingly, total fluorescence measurements provide a suitable estimate of the degree of surface functionalization produced during conjugation. We repeated the conjugation protocol using different concentrations of biotin before labeling the beads with fluorescent streptavidin. The flow cytometry measurements shown in FIG. 23B, provide the average total fluorescent signal of each bead population and conjugation condition. The fluorescent intensity varied with the concentration of biotin during conjugation, confirming this method can produce populations with various degrees of surface biotinylation. The decreasing trend in total fluorescence across bead types results from the differences in diffusion into each bead type during conjugation. These bead populations were measured by differential bead counting device determining the percent capture for each bead type and level of surface biotin. The results of these experiments are shown in FIG. 23C. When no biotin is conjugated, the capture % for each bead type was minimal (<10%). As the accessible biotin on the beads increased, the capture percentage also increased then saturates close to 100% capture demonstrating the sensitivity of capture to the surface biomolecules. We are able to access the full range of bead capture at this flow rate by varying the degree of conjugation. FIG. 25 and video capture (not shown), provide examples of bead capture; biotinylated beads can be seen sticking to a streptavidin-coated pillar as they flow through the device. Importantly, these capture experiments were performed at a single optimized flow rate (20 μL min⁻¹), confirming that the different bead types are suitable for simultaneous, biotin-sensitive capture.

Next, the selectivity of bead capture was tested by simultaneously flowing a mix of all three bead types. In each experiment one of the populations was conjugated to biotin while the others were left unconjugated. FIGS. 23D-23E show the entrance and exit histograms obtained in this way. As expected, only the MHB population that was conjugated to biotin shows a significant drop in the exit count as all 3 populations are processed simultaneously. The unconjugated beads are not captured and as a result show similar entrance and exit counts. From these experiments, we show that the electrically distinct bead populations can be captured sensitively and selectively in response to the surface biotin.

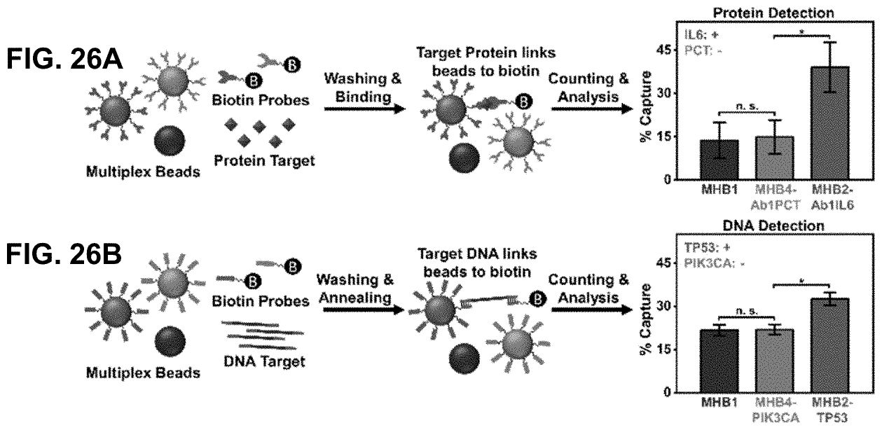

Application for DNA and Protein Monitoring. Finally, we tested and validated the new MHBs for multiplexed monitoring of biomolecules. This platform has been previously shown to sensitively detect and quantify a single protein target in a physiologically relevant range using the capture response of a single population of latex beads.²⁴ To confirm the multiplexing capabilities of the MHBs for protein detection we determined the impact of conjugation and analyte binding on the capture response of each bead (FIG. 26A-26B). MHB1 was used as a negative control and was left unconjugated to measure the background rate of nonspecific capture. MHB2 and MHB4 were separately conjugated to primary antibodies against two of the most well-studied biomarkers for sepsis, IL-6 and PCT respectively.⁴⁴⁻⁴⁷ The three bead populations were mixed with a sample containing IL-6 and biotin-tagged secondary antibodies against IL-6 and PCT. MHB2 is a positive for its analyte and forms a sandwiched complex that affixes biotin capture groups to the bead surface. MHB4 acts as a no target control. By comparing MHB1 and MHB4 we confirm that the conjugation of primary antibodies to the beads does not result in an increase in capture. Compared to MHB1 and MHB4, only MHB2 which is positive for its target analyte shows a significant increase in capture. This configuration confirms that each bead type responds independently to its target analyte which is fundamental to a multiplexed measurement. The rate of non-specific capture was found to increase slightly during these multiplexed measurements. As the total number of beads processed by the capture chamber increases, the probability of collisions between flowing beads and those that have already been captured also rises, which can result in further bead capture. This effect can be minimized by increasing the total size of the capture chamber and the inter-pillar spacing.

In order to demonstrate the versatility of the bead counting platform we also validated the MHBs for a multiplexed a DNA detection assay as shown in FIG. 26B. Using the same principles from the protein detection experiment, MHB1 was unconjugated and acts as the negative control, while MHB2 and MHB4 were separately conjugated to short (˜20 bp) DNA binding oligos complementary to unique gene fragments. The chosen sequence fragments correspond to TP53 and PIK3CA circulating tumor DNA which has been observed in elevated levels in the blood of patients with colorectal cancer.⁴⁸ After functionalization, the 3 bead populations were mixed with a sample containing TP53 fragments and biotin-tagged DNA probes against all targets. Sequence annealing results in the linkage of beads to biotin in the presence of the correct DNA fragment. As expected, the level of nonspecific capture of the two controls, MHB1 and MHB4, was similar, confirming that the conjugation of DNA targeting sequences does not result in an increased nonspecific capture response. The positive control, MHB2 was the only bead type to show significantly elevated capture in response to the presence of its target analyte. These experiments confirm the unique, analyte dependence of the capture response for each bead type. The newly designed MHBs enable a simple, multiplexed electrical method of monitoring multiple types of biomolecules by bead counting and capture.

Leveraging droplet microfluidics, we have designed, synthesized, and characterized multiple electrically distinct populations of magnetic, polyacrylamide hydrogel beads. These beads are a platform for a new method of multiplexed biomolecule measurement using only electrical detection by Coulter counting. We confirmed the sensitive and selective capture of each bead population under simultaneous flow in a microfluidic device. These newly synthesized beads overcome the challenges associated with multiplexing the bead counting device and provide a critical foundation for the development of rapid multiplexed electrical quantification of proteins and biomolecules. Importantly, we detect multiple types of biomolecules using a single versatile platform. Moreover, the magnetic properties of these beads present a new opportunity for orthogonal measurements of cells and biomolecules or for enhanced multiplexing using an additional set of non-magnetic electrically distinct hydrogels. Using these new beads, it is now possible to develop a single integrated electrical point-of-care device capable of a combined measurement of cell count, the levels of a cell surface antigen, and multiple protein concentrations for improved rapid diagnostics.

Estimation of Magnetic Nanoparticle Loading: The saturation magnetization (Ms) of each sample was measured and used to estimate the number of magnetic nanoparticles per microsphere. According to the manufacturer, the nanoparticles consist of at least 80% γ-Fe₂O₃ which has a bulk Mₛ of 76 emu g⁻¹. We assumed that the magnetic particles were uniformly incorporated and did not significantly alter the density of bulk hydrogel. As such we could use the hydrogel diameter, the nanoparticle diameter, and the dry mass of the sample to estimate the number of nanoparticles per microsphere. Accordingly, MHB1 and 3 contained on average 20 magnetic nanoparticles per microspheres while MHB2 and MHB4 contained 23 nanoparticles. This result is consistent with a Poisson loading.

### REFERENCES FOR EXAMPLE 7

1 W. J. Shen, Y. Zhuo, Y. Q. Chai, Z. H. Yang, J. Han and R. Yuan, ACS Appl. Mater. Interfaces, 2015, 7, 4127-4134.

2 V. S. P. K. S. A. Jayanthi, A. B. Das and U. Saxena, Biosens. Bioelectron., 2017, 91, 15-23.

3 D. Kruger, Y. Y. Yako, J. Devar, N. Lahoud and M. Smith, PLoS One, 2019, 14, e0221169.

4 J. J. Park, O. Harari, L. Dron, E. J. Mills and K. Thorlund, Contemp. Clin. Trials Commun., DOI:10.1016/j.conctc.2019.100396.

5 C. W. Seymour, V. X. Liu, T. J. Iwashyna, F. M. Brunkhorst, T. D. Rea, A. Scherag, G. Rubenfeld, J. M. Kahn, M. Shankar-Hari, M. Singer, C. S. Deutschman, G. J. Escobar and D. C. Angus, JAMA—J. Am. Med. Assoc., 2016, 315, 762-774.

6 D. Su, D. Zhang, J. Jin, L. Ying, M. Han, K. Chen, B. Li, J. Wu, Z. Xie, F. Zhang, Y. Lin, G. Cheng, J. Y. Li, M. Huang, J. Wang, K. Wang, J. Zhang, F. Li, L. Xiong, A. Futreal and W. Mao, Nat. Commun., 2019, 10, 5076.

7 K. Zhao, M. Tang, H. Wang, Z. Zhou, Y. Wu and S. Liu, Biosens. Bioelectron., 2019, 126, 767-772.

8 H. Peng, Z. Huang, W. Wu, M. Liu, K. Huang, Y. Yang, H. Deng, X. Xia and W. Chen, ACS Appl. Mater. Interfaces, 2019, 11, 24812-24819.

9 H. H. Dolin, T. J. Papadimos, S. Stepkowski, X. Chen and Z. K. Pan, SHOCK, 2018, 49, 364-370.

10 S. Gibot, M. C. Béné, R. Noel, F. Massin, J. Guy, A. Cravoisy, D. Barraud, M. D. C. Bittencourt, J. P. Quenot, P. E. Bollaert, G. Faure and P. E. Charles, Am. J. Respir. Crit. Care Med., 2012, 186, 65-71.

11 K. Oved, A. Cohen, O. Boico, R. Navon, T. Friedman, L. Etshtein, O. Kriger, E. Bamberger, Y. Fonar, R. Yacobov, R. Wolchinsky, G. Denkberg, Y. Dotan, A. Hochberg, Y. Reiter, M. Grupper, I. Srugo, P. Feigin, M. Gorfine, I. Chistyakov, R. Dagan, A. Klein, I. Potasman and E. Eden, PLoS One, DOI:10.1371/journal.pone.0120012.

12 L. Sun, H. Tu, T. Chen, Q. Yuan, J. Liu, N. Dong and Y. Yuan, Sci. Rep., DOI:10.1038/s41598-017-12022-1.

13 L. Tang and J. Casas, Biosens. Bioelectron., 2014, 61, 70-75.

14 H. R. Wong, S. L. Weiss, J. S. Giuliano, M. S. Wainwright, N. Z. Cvijanovich, N. J. Thomas, G. L. Allen, N. Anas, M. T. Bigham, M. Hall, R. J. Freishtat, A. Sen, K. Meyer, P. A. Checchia, T. P. Shanley, J. Nowak, M. Quasney, A. Chopra, J. C. Fitzgerald, R. Gedeit, S. Banschbach, E. Beckman, K. Harmon, P. Lahni and C. J. Lindsell, PLoS One, 2014, 9, e92121.

15 J. P. Anderson, L. N. Rascoe, K. Levert, H. M. Chastain, M. S. Reed, H. N. Rivera, I. McAuliffe, B. Zhan, R. E. Wiegand, P. J. Hotez, P. P. Wilkins, J. Pohl and S. Handali, PLoS Negl. Trop. Dis., DOI:10.1371/journal.pntd.0004168.

16 D. C. Appleyard, S. C. Chapin and P. S. Doyle, Anal. Chem., 2011, 83, 193-199.

17 L. Cohen and D. R. Walt, Chem. Rev., 2019, 119, 293-321.

18 S. X. Leng, J. E. McElhaney, J. D. Walston, D. Xie, N. S. Fedarko and G. A. Kuchel, Journals Gerontol. Ser. A Biol. Sci. Med. Sci., 2008, 63, 879-884.

19 N. Reslova, V. Michna, M. Kasny, P. Mikel and P. Kralik, Front. Microbiol., 2017, 8, 55.

20 B. S. Munge, T. Stracensky, K. Gamez, D. DiBiase and J. F. Rusling, Electroanalysis, 2016, 28, 2644-2658.

21 F. Liu, L. Ni and J. Zhe, Biomicrofluidics, DOI:10.1063/1.5022168.

22 K. R. Ludwig and A. B. Hummon, Mol. Biosyst., 2017, 13, 648-664.

23 R. Rodriguez-Trujillo, M. A. Ajine, A. Orzan, M. D. Mar, F. Larsen, C. H. Clausen and W. E. Svendsen, Sensors Actuators, B Chem., 2014, 190, 922-927.

24 E. Valera, J. Berger, U. Hassan, T. Ghonge, J. Liu, M. Rappleye, J. Winter, D. Abboud, Z. Haidry, R. Healey, N. T. Hung, N. Leung, N. Mansury, A. Hasnain, C. Lannon, Z. Price, K. White and R. Bashir, Lab Chip, 2018, 18, 1461-1470.

25 N. N. Watkins, U. Hassan, G. Damhorst, H. K. Ni, A. Vaid, W. Rodriguez and R. Bashir, Sci. Transl. Med., DOI:10.1126/scitranslmed.3006870.

26 U. Hassan, T. Ghonge, B. Reddy, M. Patel, M. Rappleye, I. Taneja, A. Tanna, R. Healey, N. Manusry, Z. Price, T. Jensen, J. Berger, A. Hasnain, E. Flaugher, S. Liu, B. Davis, J. Kumar, K. White and R. Bashir, Nat. Commun., DOI:10.1038/ncomms15949.

27 S. Murali, X. Xia, A. V. Jagtiani, J. Carletta and J. Zhe, Smart Mater. Struct., DOI:10.1088/0964-1726/18/3/037001.

28 M. Pellegrini, A. Cherukupalli, M. Medini, R. Falkowski and R. Olabisi, Tissue Eng.-Part C Methods, 2015, 21, 1246-1250.

29 D. V. Amato, H. Lee, J. G. Werner, D. A. Weitz and D. L. Patton, ACS Appl. Mater. Interfaces, 2017, 9, 3288-3293.

30 T. Femmer, A. Jans, R. Eswein, N. Anwar, M. Moeller, M. Wessling and A. J. C. Kuehne, ACS Appl. Mater. Interfaces, 2015, 7, 12635-12638.

31 M. M. Islam, A. Loewen and P. B. Allen, Sci. Rep., 2018, 8, 8763.

32 J. W. Kim, A. S. Utada, A. Fernandez-Nieves, Z. Hu and D. A. Weitz, Angew. Chemie-Int. Ed., 2007, 46, 1819-1822.

33 S. Y. Teh, R. Lin, L. H. Hung and A. P. Lee, Lab Chip, 2008, 8, 198-220.

34 D. Sehgal and I. K. Vijay, Anal. Biochem., 1994, 218, 87-91.

35 E. Valera, A. Muriano, I. Pividori, F. Sanchez-Baeza and M. P. Marco, Biosens. Bioelectron., 2013, 43, 211-217.

36 D. L. Holmes and N. C. Stellwagen, Electrophoresis, 1991, 12, 612-619.

37 B. Yang, Y. Lu and G. Luo, in Industrial and Engineering Chemistry Research, 2012, vol. 51, pp. 9016-9022.

38 O. Okay, 2009, pp. 1-14.

39 H. Morgan, D. Holmes and N. G. Green, Curr. Appl. Phys., 2006, 6, 367-370.

40 X. Cheng, D. Irimia, M. Dixon, K. Sekine, U. Demirci, L. Zamir, R. G. Tompkins, W. Rodriguez and M. Toner, Lab Chip, 2007, 7, 170-178.

41 X. Cheng, A. Gupta, C. Chen, R. G. Tompkins, W. Rodriguez and M. Toner, Lab Chip, 2009, 9, 1357-1364.

42 C. E. Chivers, A. L. Koner, E. D. Lowe and M. Howarth, Biochem. J., 2011, 435, 55-63.

43 O. H. Laitinen, V. P. Hytönen, H. R. Nordlund and M. S. Kulomaa, Cell. Mol. Life Sci., 2006, 63, 2992-3017.

44 S. Kumar, S. Tripathy, A. Jyoti and S. G. Singh, Biosens. Bioelectron., 2019, 124-125, 205-215.

45 B. Reddy, U. Hassan, C. Seymour, D. C. Angus, T. S. Isbell, K. White, W. Weir, L. Yeh, A. Vincent and R. Bashir, Nat. Biomed. Eng., 2018, 2, 640-648.

46 C. Russell, A. C. Ward, V. Vezza, P. Hoskisson, D. Alcorn, D. P. Steenson and D. K. Corrigan, Biosens. Bioelectron., 2019, 126, 806-814.

47 P. Seshadri, K. Manoli, N. Schneiderhan-Marra, U. Anthes, P. Wierzchowiec, K. Bonrad, C. Di Franco and L. Torsi, Biosens. Bioelectron., 2018, 104, 113-119.

48 F. Diehl, K. Schmidt, M. A. Choti, K. Romans, S. Goodman, M. Li, K. Thornton, N. Agrawal, L. Sokoll, S. A. Szabo, K. W. Kinzler, B. Vogelstein and L. A. Diaz, Nat. Med., 2008, 14, 985-990.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every device, system, formulation, combination of components, or method described or exemplified herein can be used to practice the invention, unless otherwise stated.

Whenever a range is given in the specification, for example, a number range, a volume range, a percentage range, a statistical parameter, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A multiplexible particle system for use with an electronic detector to detect a plurality of distinct targets comprising:
a plurality of monodisperse polymer particle populations, each population having a unique electrical parameter during flow through a spatially confined electric field for multiplexed detection; and
wherein the unique electrical parameter has an electrical parameter distribution for each polymer particle population flowing through a spatially confined electrical field of the electronic detector, and the distribution in a given polymer particle population is sufficiently narrow to minimize overlap with any other polymer particle population to achieve the multiplexed detection.

2. The multiplexible particle system of claim 1, wherein the electrical parameter is electrical impedance and the sufficiently narrow distribution is characterized by a coefficient of variation of the electrical impedance measured by the electronic detector that is less than 15%.

3. The multiplexible particle system of claim 1, wherein each polymer particle population has an average density difference and/or an average diameter difference with respect to every other polymer particle population to provide during use substantially equivalent flow properties during flow in a suspending solution; wherein:
the average density difference is within 30% of any other polymer particle population and is within 30% of a suspending solution density; and
the average diameter difference between any two populations is less than or equal to 20%.

4. (canceled)

5. The multiplexible particle system of claim 1, wherein the polymer particles comprise cross-linked monomers and/or polymers that form a meshwork scaffold having functional groups corresponding to conjugation sites.

6. The multiplexible particle system of claim 1, wherein at least one polymer particle population comprises one or more solid particles embedded in a hydrogel, wherein the solid particles:
have an average diameter that is greater than an average pore size in the polymer particle; and/or
are chemically linked to the polymer particle.

7. The multiplexible particle system of claim 6, wherein at least one solid particle is a magnetic particle.

8. (canceled)

9. The multiplexible particle system of claim 1, wherein at least one polymer particle population comprise hydrogel particles of at least 80% by weight water.

10. (canceled)

11. (canceled)

12. The multiplexible particle system of claim 1, wherein the plurality of polymer particle populations each have an average diameter that is less than 1 mm.

13. The multiplexible particle system of claim 1, wherein the spatially confined electrical field corresponds to a microchannel of the electronic device that detects particle passage by resistive pulse sensing.

14. The multiplexible particle system of claim 1, further comprising a suspending solution that is an aqueous electrolyte solution having a density of between 1 g/cm³ to 1.9 g/cm³.

15. The multiplexible particle system of claim 1, wherein the polymer particle populations are selected to each have an electrical parameter with a mean value and a standard deviation such that during use the system provides a polymer particle population electrically distinguishable error rate that is less than 10%.

16. The multiplexible particle system of claim 1, wherein the unique electrical parameter has a value based on one or more of:
polymer composition;
polymer size;
polymer density;
presence or absence of a solid particle within the polymer;
volume fraction of solid particles within the polymer;
functional groups in the polymer that affect polymer hydration status; or
organic and/or inorganic moieties attached to the polymer particles.

17. The multiplexible particle system of claim 1, wherein the polymer particle is formed from a material selected from the group consisting of:
polyacrylamide;
poly(N-isopropylacrylam ide);
alginate;
agarose;
poly(ethyleneglycol)diacrylate;
polyacrylate;
polyvinyl alcohol;
copolymers having an abundance of hydrophilic groups; and
a mixture of any two or more of the above materials.

18. The multiplexible particle system of claim 1, comprising between 2 and 100 distinct populations.

19. The multiplexible particle system of claim 1, further comprising a tag connected to and/or embedded in at least one polymer particle population to further increase multiplexing capacity, wherein the tag is selected from the group consisting of one or more of:
an optical label;
a magnetic particle;
a receptor molecule;
a target molecule; and
groups that are orthogonally reactive.

20. (canceled)

21. A method of making a plurality of electrically-distinct polymer particle populations, the method comprising the steps of:
flowing a plurality of unique pre-polymer solutions and an immiscible fluid through a microfluidic drop-making junction to form a plurality of liquid droplets suspended in the immiscible fluid;
providing a surfactant to the plurality of liquid droplets suspended in the immiscible fluid;
polymerizing the pre-polymer solutions in the plurality of liquid droplets suspended in immiscible fluid;
breaking and opening the plurality of polymerized liquid droplets to disperse a plurality of monodisperse polymer particle populations into an aqueous solution; and
wherein each population has a distinct electrical impedance signature when flowing through a spatially confined electrical field.

22. The method of claim 21, further comprising the step of selecting each of the plurality of pre-polymer solutions and/or further processing at least one polymerized polymer particle population; to generate the unique electrical impedance signature during flow through the spatially confined electrical field, wherein each polymer particle population is characterized by a coefficient of variation of the measured electrical impedance that is less than 15%.

23-30. (canceled)

31. A method of detecting a target molecule, the method comprising the steps of:
providing the multiplexible polymer particle system of claim 1;
conjugating each polymer particle population with a unique target detection molecule;
contacting the plurality of polymer particle populations with a sample comprising a target molecule that specifically binds to a specific target detection material, thereby binding the target molecule to the polymer particle;
flowing the polymer particle populations past an entrance detector;
counting the number of polymer particles in each population that pass the first detector;
selectively capturing polymer particles in a capture chamber;
flowing the polymer particles not captured in the capture chamber past an exit detector;
counting the number of polymer particles in each population that pass the exit detector;
determining the difference in flowing polymer particles past the entrance and exit detector, thereby detecting the target molecule.

32. The method of claim 31, wherein the method is a multiplex method for detecting two or more target molecules in a single run, the method further comprising the step of:
identifying the polymer particle population of a polymer particle that passes the detectors by measuring the unique electrical parameter as the particle flows past the detector.

33. The method of claim 31, further comprising determining a concentration of target molecules in the sample by:
obtaining a calibration curve;
measuring the amount of a captured particle population; and
determining from the amount of captured particle population and the calibration curve the concentration of target molecules in the sample.

34. The method of claim 31, wherein the target molecule is selected from the group consisting of:
a DNA sequence;
an RNA sequence;
an amino acid sequence;
a cell surface protein;
a protein biomarker from a biological sample; and
chemical moieties that are orthogonally reactive to functional groups of the polymer particles.

35. The method of claim 31, further comprising the step of:
tuning a surface functional group density to optimize detection sensitivity over a target molecule concentration range.

36. (canceled)

37. (canceled)

38. The method of claim 31, further comprising the steps of:
magnetically capturing particle populations having a magnetizable particle in the polymer; and later releasing the magnetically captured particle populations, thereby increasing multiplexing.

39. (canceled)

40. A method of encoding a fluid material identity, the method comprising the steps of:
introducing the multiplexible particle system of claim 1 to a fluid material, wherein the ratio or presence of each polymer particle population is known, thereby encoding the fluid material identity.

41-48. (canceled)

49. A highly multiplexed detection method, the method comprising the steps of:
providing a plurality of polymer particle populations, wherein each population has an electrical signature and at least one population has a degradation parameter;
first flowing the plurality of polymer particle populations through a confined electric field and measuring the electrical signature of the polymer particles passing the confined electric field;
applying a degradation stimulus to the polymer particles that have passed the confined electric field, wherein the degradation stimulus is targeted to at least one degradation parameter, thereby degrading polymer particle populations having the targeted degradation parameter to generate a degraded plurality of polymer particle populations; and
second flowing the degraded plurality of polymer particle populations through a confined electric field and measuring the electrical signature of the polymer particles passing the confined electric field.

50. The method of claim 49, wherein the degradation stimulus is selected from the group consisting of:
a chemical stimulus;
a biological stimulus;
a temperature stimulus;
a pH stimulus; and
an electromagnetic stimulus.

51. (canceled)

52. (canceled)
